(19) United States
(12) Patent Application Publication
(10) Pub. No.: US 2026/0258089 A1
(43) Pub. Date: Sep. 3, 2026

(54) METHOD FOR SCREENING BIOLOGICALLY RELEVANT SULFO DONOR AND ACCEPTOR SUBSTRATES FOR ARYL-SULFATE SULFOTRANSFERASES

(71) Applicants: Dhara D. SHAH, Chandler, AZ (US); Rosa KRAJMALNIK-BROWN, Chandler, AZ (US)

(72) Inventors: Dhara D. SHAH, Chandler, AZ (US); Rosa KRAJMALNIK-BROWN, Chandler, AZ (US)

(21) Appl. No.: 19/551,906

(22) Filed: Feb. 27, 2026

Related U.S. Application Data
(60) Provisional application No. 63/764,872, filed on Feb. 28, 2025.

(51) Int. Cl. C07K 14/005

(52) U.S. Cl. CPC C07K 14/005

(57) ABSTRACT

Microbial aryl-sulfate sulfotransferases (ASSTs), catalyze sulfo transfer reactions, part of phase II detoxification pathways, without utilizing 3′-phosphate-5′-phosphosulfate (PAPS) as a donor. An exemplary sulfotransferase (Bacteroides vulgatus ASST, or BvASST) from the prevalent gut microbe B. vulgatus (now Phocaeicola vulgatus) is a versatile catalyst that utilizes a wide range of phenolic molecules as substrates that are commonly encountered by the host. With this action, ASSTs modulate concentrations of donor phenolic sulfates such as acetaminophen sulfate, dopamine sulfate, p-cresol sulfate, and related compounds and display broad acceptor flexibility by sulfonating diverse phenolic compounds, including p-coumaric acid, p-cresol, 4-ethylphenol, tyramine, among others. Gut microbial enzymes like ASSTs may contribute to host detoxification of phenolics, a role previously attributed solely to human sulfotransferases.

### CROSS-REFERENCE TO RELATED APPLICATIONS

This application claims priority to U.S. Provisional Patent Application No. 63/764,872, filed on Feb. 28, 2025, which is incorporated by reference herein in its entirety.

### REFERENCE TO SEQUENCE LISTING

This application was filed with a Sequence Listing XML in ST.26 XML format in accordance with 37 C.F.R. § 1.831. The Sequence Listing XML file submitted in the USPTO Patent Center, “208192-0038-US02_sequence_listing_xml_20 Feb. 2026.xml,” was created on Feb. 20, 2026, contains 14 sequences, has a file size of 19.9 kilobytes (20,400 bytes), and is incorporated by reference in its entirety into the specification.

### BACKGROUND

Sulfonation is a chemical process that helps remove harmful compounds from human bodies. While human enzymes that do this are well known, similar enzymes from gut bacteria are not well understood. Understanding these bacterial enzymes could change how we view drug metabolism and the role of gut bacteria in health.

What is needed are enzymes that can transfer sulfo groups between different phenolic molecules and methods for identifying biologically relevant sulfo donor and acceptor substrates for aryl-sulfate sulfotransferases.

### SUMMARY

One embodiment described herein is a method for identifying sulfo donor substrates for an aryl-sulfate sulfotransferase (ASST), the method comprising: providing an ASST having an unsulfonated form; incubating the ASST with a candidate sulfo donor compound; and analyzing the ASST by intact protein mass spectrometry to detect conversion of the unsulfonated form to a sulfonated form, thereby identifying the candidate sulfo donor compound as a sulfo donor substrate for the ASST. In one aspect, the aryl-sulfate sulfotransferase is a PAPS independent sulfotransferase. In another aspect, the unsulfonated form has a first molecular mass; the sulfonated form has a second molecular mass that is approximately 80 Da greater than the first molecular mass; and detecting the second molecular mass during the analyzing step identifies the candidate sulfo donor compound as a sulfo donor substrate for the ASST. In another aspect, the candidate sulfo donor compound comprises a phenolic sulfate or a non-phenolic sulfate. In another aspect, the candidate sulfo donor compound comprises acetaminophen sulfate, dopamine sulfate, p-coumaric acid sulfate, p-cresol sulfate, indoxyl sulfate, 4-methylumbelliferyl sulfate, or p-nitrophenyl sulfate. In another aspect, the method further comprises isolating the ASST prior to the analyzing step. In another aspect, isolating the ASST comprises heterologous expression in E. coli, purification via column chromatography, and spin filtering. In another aspect, the ASST is a Bacteroides vulgatus ASST. In another aspect, the ASST has at least 90-99% identity to a polypeptide sequence selected from SEQ ID NO: 5-7. In another aspect, the ASST has a polypeptide sequence selected from SEQ ID NO: 5-7.

Another embodiment described herein is a method for identifying sulfo acceptor substrates for an aryl-sulfate sulfotransferase (ASST), the method comprising: providing a sulfonated form of an ASST; incubating the sulfonated form of the ASST with a candidate sulfo acceptor compound; and analyzing the ASST by intact protein mass spectrometry to detect conversion of the sulfonated form to an unsulfonated form, thereby identifying the candidate sulfo acceptor compound as a sulfo acceptor substrate for the ASST. In one aspect, the aryl-sulfate sulfotransferase is a PAPS independent sulfotransferase. In another aspect, the sulfonated form has a first molecular mass; the unsulfonated form has a second molecular mass that is approximately 80 Daless than the first molecular mass; and detecting the second molecular mass during the analyzing step identifies the candidate sulfo acceptor compound as a sulfo acceptor substrate for the ASST. In another aspect, the candidate sulfo donor compound comprises a phenolic sulfate or a non-phenolic sulfate. In another aspect, the method further comprises isolating the ASST prior to the analyzing step. In another aspect, isolating the ASST comprises heterologous expression in E. coli, purification via column chromatography, and spin filtering. In another aspect, the method further comprises: incubating the ASST in the unsulfonated form with a sulfo group donor, thereby converting it to the sulfonated form; and isolating the sulfonated form of the ASST; wherein incubating the ASST in the unsulfonated form with the sulfo group donor and isolating the sulfonated form are performed prior to providing the sulfonated form of the ASST. In another aspect, the sulfo group donor is selected from the group consisting of acetaminophen sulfate, dopamine sulfate, p-coumaric acid sulfate, p-cresol sulfate, indoxyl sulfate, 4-methylumbelliferyl sulfate, and p-nitrophenyl sulfate. In another aspect, the ASST is a Bacteroides vulgatus ASST. In another aspect, the ASST has at least 90-99% identity to a polypeptide sequence selected from SEQ ID NO: 5-7. In another aspect, the ASST has a polypeptide sequence selected from SEQ ID NO: 5-7.

Another embodiment described herein is a modified Bacteroides vulgatus ASST having at least 90-99% identity to a polypeptide sequence selected from SEQ ID NO: 5-7. In one aspect, the ASST has a polypeptide sequence selected from SEQ ID NO: 5-7. In another aspect, the modified Bacteroides vulgatus ASST further comprising a PelB leader sequence or a signal peptide.

### DESCRIPTION OF THE DRAWINGS

FIG. 1 shows a schematic of the general reaction catalyzed by aryl-sulfate sulfotransferases (ASSTs).

FIG. 2 shows the predicted sulfonation state of Bacteroides vulgatus (now recategorized as Phocaeicola vulgatus) aryl-sulfate sulfotransferase (BvASST) throughout the course of the reaction.

FIG. 3A-K show that BvASST undergoes sulfonation by several phenolic sulfates. Intact protein mass spectra of sulfonated (([M+1]+80)) and unsulfonated ([M+1]) forms of BvASST with various donors are shown. In each panel, the black, baseline spectrum represents BvASST without the addition of the donor substrate, which contains a fraction of enzyme in a sulfonated form. The lighter color spectrum in each panel represents mass spectrum of intact BvASST after the incubation with 100 μM of donor and the darker shade represents mass spectrum of intact BvASST after the incubation with 1 mM of donor. All incubations were carried out at 37° C. for 20 min. FIG. 3A shows BvASST spectra captured with 4-MUS. FIG. 3B shows BvASST spectra captured with pCAS. FIG. 3C shows BvASST spectra captured with pNPS. FIG. 3D shows BvASST spectra captured with acetS. FIG. 3E shows BvASST spectra captured with indoxylS. FIG. 3F shows BvASST spectra captured with pCS. FIG. 3G shows BvASST spectra captured with dopamineS. FIG. 3H shows BvASST spectra captured with serotoninS. FIG. 3I shows BvASST spectra captured with PAPS. FIG. 3J shows a schematic of qualitative trends in enzyme sulfonation across various donors and concentrations where colors and concentrations correspond to spectra shown in FIG. 3A-I, estimated as a percentage of ratio: ([M+1]+80)/([M+1]+([M+1]+80]))×100 or (sulfonated BvASST/[sulfonated+unsulfonated] BvASST)×100 (Tables 3-4). Here, 4-MUS is 4-methylumbelliferyl sulfate, pCAS is p-coumaric acid sulfate, pNPS is p-nitrophenyl sulfate, acetaminophenS is acetaminophen sulfate, indoxylS is indoxyl sulfate, p-cresolS is p-cresol sulfate, dopamineS is dopamine sulfate, and serotoninS is serotonin sulfate. FIG. 3K shows a zoomed-in baseline spectrum of purified BvASST depicting two peaks without any substrate. The major peak with molecular mass of 68,267 Da represents unsulfonated ([M+1]) form of BvASST whereas the minor peak (≈16%) with molecular mass of 68,347 Da represents sulfonated ([M+1]+80]) form of BvASST.

FIG. 4A-B show how incubation of sulfonated BvASST with various phenolic molecules reverted it to a largely unsulfonated form, confirming these compounds as compatible sulfo group acceptors. Purified BvASST (no donor or acceptor) incubated with pNPS (20 min) displayed a ([M+1]+80) Da mass shift (only donor, no acceptor). This sulfonated BvASST, when subsequently incubated with a sulfo acceptor (2 h), showed a mass shift back to its original mass [M+1]. FIG. 4A shows an intact mass spectra collected from the incubation of sulfonated BvASST (generated via incubation with pNPS) with each acceptor molecule tested. Controls do not have acceptors added and include: (i) no donor or acceptor which depicts the purified form of BvASST and (ii) incubation with pNPS (sulfo donor) only without any acceptor. FIG. 4B shows a schematic of qualitative trends for the change in BvASST sulfonation in the presence of compatible phenolic acceptor molecules where colors correspond to spectra shown in A, estimated as a percentage of ratio: ([M+1]+80)/([M+1]+([M+1]+80]))×100 or (sulfonated BvASST/[sulfonated+unsulfonated] BvASST)×100).

FIG. 5 shows the identification of the site of sulfonation for BvASST as His471. The figure depicts EThcD-MS/MS spectrum of trypsin-generated peptide fragment 460-490. Specific peptide fragments observed in the spectrum are annotated in both the spectrum and the peptide map above it where C(am) indicates alkylated (carbamidomethylated) Cys, which was generated during alkylation of reduced cysteine residues with iodoacetamide. H(sulfo) indicates sulfonated His. Fragments labeled b or c contain the N-terminus and fragments labeled y or z contain the C-terminus. From the N-terminal side, the observation of the c10+ ion shown indicates that the first 10 residues are unmodified (other than intentionally alkylated Cys). Observation of the mass-shifted c12+ ion shown indicates that either the C(am) or His(sulfo) residue must contain the sulfonated residue. Since alkylated Cys is not a candidate for sulfonation, the logical conclusion is that the His residue is sulfonated. Similar logic can be applied to the z-ion series shown. A zoomed-in spectrum of the c12+ fragment and a table of fragment ion assignments are provided in FIG. 15 and Table 1.

FIG. 6A-B show pairs of substrates (donors and acceptors) leading to productive turnovers via BvASST-catalyzed sulfo transfer reactions. Assays containing various pairs of sulfo group donor and sulfo group acceptor in the presence and absence of BvASST were incubated for 4 h at 37° C. After incubations, all assays were analyzed via HPLC to detect products that include sulfated forms of acceptors and desulfated donors which were verified by comparing their retention times to commercial authentic standards. Each substrate pair was replicated three times (n=3; FIG. 18-23. FIG. 6A shows tested donor and acceptor substrate pair reactions analyzed in terms of generation of products to determine the reaction occurrence. Ught blue squares indicate detection of both products in reactions catalyzed by BvASST, dark blue squares indicate no product detection, gray squares indicate inability to separate peaks and therefore the presence of products can't be determined (ND), and white represents self with self-type reactions (e.g., acetS with acetaminophen). All compounds were identified as compared to the retention times of commercially available standards for verification. Here, pNPS is p-nitrophenyl sulfate, pCAS is p-coumaric acid sulfate, p-cresolS is p-cresol sulfate, dopamineS is dopamine sulfate, and acetaminophenS is acetaminophen sulfate. FIG. 6B shows a representative chromatogram depicting a reaction utilizing acetS as a donor and pC as an acceptor. In the presence of BvASST, pCS and acetaminophen were produced. In the control reaction, a small peak of acetaminophen was detected due to noncatalytic hydrolysis of small percentage of acetS (FIG. 24A), but we do not detect any pCS. A reaction is only positive when both products are detected.

FIG. 7A-B show enzymatic assays to test sulfotransferase activity of BvASST with a variety of phenolic acceptor molecules. FIG. 7A shows two types of continuous enzymatic assays, a colorimetric assay with pNPS as a sulfo donor and a fluorescence-based assay with 4-MUS as a sulfo donor. FIG. 7B shows the structures of molecules tested as acceptors. Blue molecules represent sulfo donors used in kinetic assays depicted in FIG. 7A.

FIG. 8 shows that reaction velocities are proportional to BvASST concentrations for many phenolic acceptors. Initial velocities (V0) were measured for BvASST-catalyzed reactions with pNPS as a donor in the presence of various acceptors, as listed above each dataset (n=2; Table 5). The production of pNP was monitored over time (150 min) from reaction progress curves and initial datapoints were fitted with linear regression to calculate initial velocities. For each acceptor, the shade of the color from light to dark illustrates the increase in BvASST concentration from 0 to 8 μM (0, 2, 4, and 8 μM). Error bars represent ±SD.

FIG. 9 shows that various gut microbes have either single or multiple annotated genes for aryl-sulfate sulfotransferases. Genes annotated to encode aryl-sulfate sulfotransferases (asst) were retrieved for common commensal gut microbes from available genome databases. The heatmap shows number of annotated asst genes within a single strain's genome sorted from highest to lowest within a selected gut commensals and various E. coli strains.

FIG. 10A-B show an ASST construct with signal peptide and pelB sequence. FIG. 10A shows the amino acid sequence of ASST protein (SEQ ID NO: 5) highlighting PelB leader sequence (red) and a signal peptide (blue). Expected molecular mass is 70,763 Da. FIG. 10B shows an SDS-PAGE to analyze ASST expression. Protein band with an expected mass is visible in fractions labeled post IPTG induction, sonicated sample (whole, before separating pellet and supernatant), and in sonicated pellet. Protein band with an expected mass is not visible in sonication supernatant, indicating its presence within cell debris. In addition to undiluted samples, 1/10th diluted and 10 times (10×) concentrated samples were also analyzed. This form of ASST with an in-tact signal peptide was not amenable to purification.

FIG. 11A-C show that BvASST is a lipoprotein. FIG. 11A shows that data generated by SignalP-6.0 shows predicted signal peptide likelihood for full length BvASST. FIG. 11B shows a schematic representing the architecture and possible attachment site of BvASST. FIG. 11C shows a table depicting experimentally measured protein solubility for BvASST with various tags.

FIG. 12A-C show an ASST construct with a pelB sequence and without a signal peptide. FIG. 12A shows the amino acid sequence of ASST (SEQ ID NO: 6) with the pelB leader sequence shown in red. Expected molecular weight 68,266 Da. FIG. 12B shows the observed molecular weight [M+1]: 68,267 Da, confirmed by intact protein mass spectrometry. FIG. 12C shows an SDS-PAGE of purification fractions showing a protein band at the expected mass. Lanes include samples of pre- and post-induction (IPTG), sonication (Son super), ammonium sulfate precipitation (AS super and AS pellet), and the anion-exchange chromatography steps (load flow-through, wash, and elution). Elution fractions F36 (Peak 1) and F80 (Peak 2) correspond to the center fractions of the two elution peaks.

FIG. 13A-C show an ASST construct without a signal peptide and without a pelB sequence. FIG. 13A shows the amino acid sequence of ASST without either leader sequences (SEQ ID NO: 7). Expected molecular weight: 66,055 Da. FIG. 13B shows the intact mass data confirmed molecular weight after methionine loss, M-met, [[M+1]−131 Da]. FIG. 13C shows an SDS-PAGE with various fractions from protein purification showing a protein band with an expected mass. Wells show protein before and after induction, sonication, ammonium sulfate precipitation, load, wash, and elution from an anion exchange column.

FIG. 14A-B show that ASST activity remains the same with or without pelB leader sequence. FIG. 14A shows a comparison of enzyme activity through product (pNP) formation using enzyme with pelB, without pelB, and a control without enzyme. Assays contained 1 mM p-nitrophenyl sulfate (pNPS), 1 mM p-coumaric acid (pCA), and 4 μM BvASST. All reactions were incubated for 1 hour at 37° C. FIG. 14B shows the mean and standard deviation of pNP production for assay performed in a (n=3).

FIG. 15 shows C12 fragmentation of sulfonated ASST. C12 shows the key sulfonated fragment with its isotopes identified by LC-MS/MS on an Orbitrap Fusion Lumos.

FIG. 16 shows that a catalytic histidine residue that undergoes sulfonation is conserved between E. coli CFT073 (His 436) and B. vulgatus (His 471) ASSTs. Sulfonated amino acid residue is identified at His 436 in E. coli ASST and His 471 in BvASST without signal peptide and harboring intact pelB. This residue corresponds to His 449 in the construct without signal peptide or pelB, and His 492 in construct with both signal peptide and pelB. Sequences are SEQ ID NO: 8-12 for the Consensus, EcASST—sp; Bv ASST—sp—pelb; Bv ASST—sp+pelb; and Bv ASST+sp+pelb, respectively.

FIG. 17A-C show preliminary assay examples with synthetic sulfo donors. FIG. 17A shows results of an assay where reactions were incubated with 500 μM pNPS, 15 μM ASST, and increasing concentrations of tyramine to test initial activity. Assays were incubated at 37° C. and measured spectrophotometrically as pNP production (absorbance at 405 nm ε=18,000 M−1 cm−1). FIG. 17B shows results of an assay using the same method as FIG. 17A, with 2000 μM pNPS.

FIG. 17C shows results of an assay where reactions used 50 μM of 4-MUS as a synthetic sulfo donor, 5 μM ASST, and 50 μM of various sulfo acceptors to test initial activity. Assays were measured at 37° C. and quantified as 4-MU production (460 nm excitation/360 nm emission).

FIG. 18 shows HPLC chromatograms for reactions of p-nitrophenyl sulfate as a donor with various acceptors for results presented in FIG. 6. Assays containing 1 mM p-nitrophenyl sulfate, 1 mM acceptor, and 4 μM BvASST were analyzed via HPLC to check the presence of sulfated acceptor and desulfated donor—two reaction products, indicating sulfotransfer reactions. Presence of products was confirmed with commercial standards and controls lacking BvASST.

FIG. 19 shows HPLC chromatograms for reactions of p-coumaric acid sulfate as a donor with various acceptors for results presented in FIG. 6. Assays containing 1 mM p-coumaric acid sulfate, 1 mM acceptor, and 4 μM BvASST were analyzed via HPLC to check the presence of sulfated acceptor and desulfated donor—two reaction products, indicating sulfotransfer reactions. Presence of products was confirmed with commercial standards and controls lacking BvASST.

FIG. 20 shows HPLC chromatograms for reactions of p-cresol sulfate as a donor with various acceptors for results presented in FIG. 6. Assays containing 1 mM p-cresol sulfate, 1 mM acceptor, and 4 μM BvASST were analyzed via HPLC to check the presence of sulfated acceptor and desulfated donor—two reaction products, indicating sulfotransfer reactions. Presence of products was confirmed with commercial standards and controls lacking BvASST.

FIG. 21 shows HPLC chromatograms for reactions of dopamine sulfate as a donor with various acceptors for results presented in FIG. 6. Assays containing 1 mM dopamine sulfate, 1 mM acceptor, and 4 μM BvASST were analyzed via HPLC to check the presence of sulfated acceptor and desulfated donor—two reaction products, indicating sulfotransfer reactions. Presence of products was confirmed with commercial standards and controls lacking BvASST.

FIG. 22 shows HPLC chromatograms for reactions of acetaminophen sulfate as a donor with various acceptors for results presented in FIG. 6. Assays containing 1 mM acetaminophen sulfate, 1 mM acceptor, and 4 μM BvASST were analyzed via HPLC to check the presence of sulfated acceptor and desulfated donor—two reaction products, indicating sulfotransfer reactions. Presence of products was confirmed with commercial standards and controls lacking BvASST.

FIG. 23 shows HPLC chromatograms for reactions of PAPS as a donor with various acceptors for results presented in FIG. 6. Assays containing 1 mM PAPS, 1 mM acceptor, and 4 μM BvASST were analyzed via HPLC to check the presence of sulfated acceptor and desulfated donor—two reaction products, indicating sulfotransfer reactions. Presence of products was confirmed with commercial standards and controls lacking BvASST.

FIG. 24A-B show that sulfate loss depends on the stability of the compound, but BvASST does not lose sulfate from active site following incubation times. FIG. 24A shows the percentage of free sulfate lost in comparison to known concentration of substrate added. After 4 hours of incubation, multiple sulfated compounds were measured via IC to determine free sulfate in the supernatant. Free sulfate is produced through the degradation of the molecule. FIG. 246B shows the percent sulfate lost from 50 μM BvASST after 4 hours of incubation at 37° C. Measured as free sulfate in the supernatant (5 kDa spin filter) via IC analysis. Statistical analysis done in GraphPad Prism using the Kruskal-Wallis Test (p=0.7).

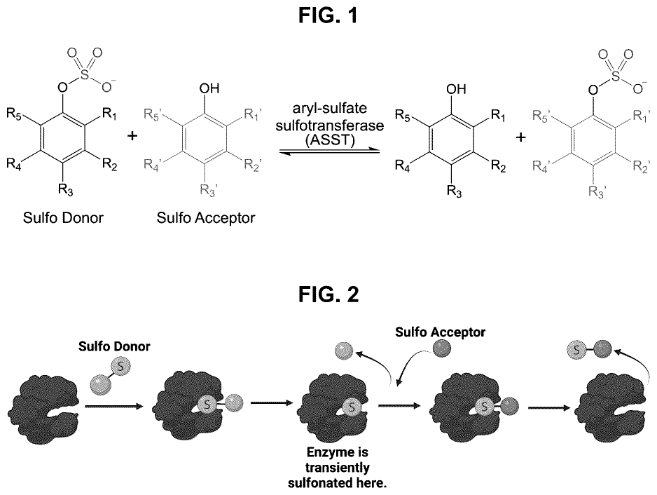

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, and chemistry described herein are well known and commonly used in the art. In case of conflict, the present disclosure, including definitions, will control the interpretation of terms. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points, or as described above in the definition of “about.”

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30° C., including all integers and endpoints within the specified range. In another aspect, “room temperature” refers a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ~20° C., ~22° C., ~25° C., or ~27° C., at standard atmospheric pressure.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein the terms “aryl-sulfate sulfotransferase” or “ASST” refer to enzymes (EC 2.8.2.22) that are capable of catalyzing a reaction between an aryl sulfate moiety and a target moiety, where the sulfate is transferred to the target moiety. These enzymes are involved in detoxification of compounds by bacteria. In one aspect, the aryl-sulfate sulfotransferase is a 3′-phosphate-5′-phosphosulfate (PAPS) independent sulfotransferase. In another aspect, the target compound has a phenolic moiety. In another aspect, the target compound has a non-phenolic moiety. In one aspect described herein, the aryl-sulfate sulfotransferase is from Bacteroides vulgatus (or Phocaeicola vulgatus) and is termed “BvASST.”

As used herein the term “Bv” refers to the bacterial species Bacteroides vulgatus which has been recategorized as Phocaeicola vulgatus. Thus, the terms “Bv” or “Bacteroides vulgatus” also refer to Phocaeicola vulgatus as used herein.

Described herein are results showing that BvASST utilizes several ASD-linked phenolic metabolites as substrates in vitro, revealing their potential role in shaping host phenolic metabolism. After investigating substrate identity and specificity, we detected a sulfonated intermediate form of BvASST in the presence of phenolic sulfo donors identified in this study and determined the site of sulfonation to be His471 consistent with previously studied Escherichia coli ASST. We also showed that the stable sulfonated form of BvASST transfers the sulfo group to various phenolic acceptors and reverts to its native unsulfonated state, enabling subsequent rounds of catalysis. Together, these findings define the complete catalytic cycle of BvASST using the newly identified donor and acceptor substrates. The identities of substrates elucidated in this study provide the basis for understanding potential native reactions catalyzed by ASSTs in their natural ecosystem.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% or 100% identical to (a) nucleotide sequences, or degenerate, homologous, or codon-optimized variants thereof, encoding polypeptides having the amino acid sequences in SEQ ID NO: 2 or 4-7; (b) nucleotide sequences, or degenerate, homologous, or codon-optimized variants thereof, encoding polypeptides having the amino acid sequences in SEQ ID NO: 2 or 4-7; and (c) nucleotide sequences capable of hybridizing to the complement of any of the nucleotide sequences in (a) or (b) above and capable of expressing functional polypeptides of amino acid sequences in SEQ ID NO: 2 or 4-7.

By a polynucleotide having a nucleotide sequence at least, for example, 90-99% “identical” to a reference nucleotide sequence encoding an ASST is intended that the nucleotide sequence of the polynucleotide be identical to the reference sequence except that the polynucleotide sequence can include up to about 10 to 1 point mutations, additions, or deletions per each 100 nucleotides of the reference nucleotide sequence encoding the ASST.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2: 4 82-489 (1981). This algorithm can be extended to use with peptide sequences using the scoring matrix developed by Dayhoff, Atlas of Protein Sequences and Structure, Dayhoff ed., 5 Suppl. 3: 353-358, National Biomedical Research Foundation, Washington, D.C., USA, and normalized by Gribskov, Nucl. Acids Res. 14(6): 6745-6763 (1986).

For example, due to the degeneracy of the genetic code, one having ordinary skill in the art will recognize that a large number of the nucleic acid molecules having a sequence at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequence shown in SEQ ID NO: 1 or 3, or degenerate, homologous, or codon-optimized variants thereof, will encode a functional ASST.

The polynucleotides described herein include those encoding mutations, variations, substitutions, additions, deletions, and particular examples of the polypeptides described herein. For example, guidance concerning how to make phenotypically silent amino acid substitutions is provided in Bowie et al., Science 247: 1306-1310 (1990), wherein the authors indicate that proteins are surprisingly tolerant of amino acid substitutions.

Thus, fragments, derivatives, or analogs of the polypeptides of SEQ ID NO: 2 or 4-7 can be (i) ones in which one or more of the amino acid residues (e.g., 1, 2, 3, 4, 5, 7, 8, 9, 10, 11, 12, 13, 14, 15, 16, 17, 18, 19, 20, 25, 30, 35, 40, 45, or 50 residues, or even more) are substituted with a conserved or non-conserved amino acid residue (preferably a conserved amino acid residue). Such substituted amino acid residues may or may not be one encoded by the genetic code, or (ii) ones in which one or more of the amino acid residues includes a substituent group (e.g., 1, 2, 3, 4, 5, 7, 8, 9, 10, 15, 20, 25, 30, 35, 40, 45, or 50 residues or even more), or (iii) ones in which the mature polypeptide is fused with another polypeptide or compound, such as a compound to increase the half-life of the polypeptide (for example, polyethylene glycol), or (iv) ones in which the additional amino acids are fused to the mature polypeptide, such as an IgG Fc fusion region peptide or leader or secretory sequence or a sequence which is employed for purification of the mature polypeptide or a proprotein sequence. Such fragments, derivatives, and analogs are deemed to be within the scope of those skilled in the art from the teachings herein.

In addition, fragments, derivatives, or analogs of the polypeptides of SEQ ID NO: 2 or 4-7 can be substituted with one or more conserved or non-conserved amino acid residue (preferably a conserved amino acid residue). In some cases these polypeptides, fragments, derivatives, or analogs thereof will have a polypeptide sequence at least 50%, 55%, 60%, 65%, 70%, 75%, 80%, 85%, 90%, 91%, 92%, 93%, 94%, 95%, 96%, 97%, 98%, 99% or 100% identical to the polypeptide sequence shown in SEQ ID NO: 2 or 4-7 and will comprise functional or non-functional proteins or enzymes. Similarly, additions or deletions to the polypeptides can be made either at the N- or C-termini or within non-conserved regions of the polypeptide (which are assumed to be non-critical because they have not been photogenically conserved).

As described herein, in many cases the amino acid substitutions, mutations, additions, or deletions are preferably of a minor nature, such as conservative amino acid substitutions that do not significantly affect the folding or activity of the protein or additions or deletions to the N- or C-termini. Of course, the number of amino acid substitutions, additions, or deletions a skilled artisan would make depends on many factors, including those described herein. Generally, the number of substitutions, additions, or deletions for any given polypeptide will not be more than about 100, 90, 80, 70, 60, 50, 40, 30, 25, 20, 19, 18, 17, 16, 15, 14, 13, 12, 11, 10, 9, 8, 7, 5, 6, 4, 3, 2, or 1.

Another embodiment described herein is a polynucleotide vector comprising one or more nucleotide sequences described herein.

Another embodiment described herein is a cell comprising one or more nucleotide sequences described herein or a polynucleotide vector described herein.

Another embodiment is a polypeptide encoded by a nucleotide sequence described herein. In one aspect, the polypeptide has 85% to 99% identity to SEQ ID NO: 2 or 4-7. In another aspect, the polypeptide is selected from SEQ ID NO: 2 or 4-7.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a nucleotide sequence or a polypeptide encoded by the nucleotide sequence produced by the method or the means described herein

Another embodiment described herein is the use of an effective amount of a polypeptide encoded by one or more of the nucleotide sequences described herein in SEQ ID NO: 1 or 3.

Another embodiment described herein is a research tool comprising a polypeptide encoded by a nucleotide sequence described herein.

Another embodiment described herein is a reagent comprising a polypeptide encoded by a nucleotide sequence described herein.

One embodiment described herein is a method for identifying sulfo donor substrates for an aryl-sulfate sulfotransferase (ASST), the method comprising: providing an ASST having an unsulfonated form; incubating the ASST with a candidate sulfo donor compound; and analyzing the ASST by intact protein mass spectrometry to detect conversion of the unsulfonated form to a sulfonated form, thereby identifying the candidate sulfo donor compound as a sulfo donor substrate for the ASST. In one aspect, the aryl-sulfate sulfotransferase is a 3′-phosphate-5′-phosphosulfate (PAPS) independent sulfotransferase. In another aspect, the unsulfonated form has a first molecular mass; the sulfonated form has a second molecular mass that is approximately 80 Da greater than the first molecular mass; and detecting the second molecular mass during the analyzing step identifies the candidate sulfo donor compound as a sulfo donor substrate for the ASST. In another aspect, the candidate sulfo donor compound comprises a phenolic sulfate or a non-phenolic sulfate. In another aspect, the candidate sulfo donor compound comprises acetaminophen sulfate, dopamine sulfate, p-coumaric acid sulfate, p-cresol sulfate, indoxyl sulfate, 4-methylumbelliferyl sulfate, or p-nitrophenyl sulfate. In another aspect, the method further comprises isolating the ASST prior to the analyzing step. In another aspect, isolating the ASST comprises heterologous expression in E. coli, purification via column chromatography, and spin filtering. In another aspect, the ASST is a Bacteroides vulgatus ASST. In another aspect, the ASST has at least 90-99% identity to a polypeptide sequence selected from SEQ ID NO: 5-7. In another aspect, the ASST has a polypeptide sequence selected from SEQ ID NO: 5-7.

Another embodiment described herein is a method for identifying sulfo acceptor substrates for an aryl-sulfate sulfotransferase (ASST), the method comprising: providing a sulfonated form of an ASST; incubating the sulfonated form of the ASST with a candidate sulfo acceptor compound; and analyzing the ASST by intact protein mass spectrometry to detect conversion of the sulfonated form to an unsulfonated form, thereby identifying the candidate sulfo acceptor compound as a sulfo acceptor substrate for the ASST. In one aspect, the aryl-sulfate sulfotransferase is a 3′-phosphate-5′-phosphosulfate (PAPS) independent sulfotransferase. In another aspect, the sulfonated form has a first molecular mass; the unsulfonated form has a second molecular mass that is approximately 80 Da less than the first molecular mass; and detecting the second molecular mass during the analyzing step identifies the candidate sulfo acceptor compound as a sulfo acceptor substrate for the ASST. In another aspect, the candidate sulfo donor compound comprises a phenolic sulfate or a non-phenolic sulfate. In another aspect, the method further comprises isolating the ASST prior to the analyzing step. In another aspect, isolating the ASST comprises heterologous expression in E. coli, purification via column chromatography, and spin filtering. In another aspect, the method further comprises: incubating the ASST in the unsulfonated form with a sulfo group donor, thereby converting it to the sulfonated form; and isolating the sulfonated form of the ASST; wherein incubating the ASST in the unsulfonated form with the sulfo group donor and isolating the sulfonated form are performed prior to providing the sulfonated form of the ASST. In another aspect, the sulfo group donor is selected from the group consisting of acetaminophen sulfate, dopamine sulfate, p-coumaric acid sulfate, p-cresol sulfate, indoxyl sulfate, 4-methylumbelliferyl sulfate, and p-nitrophenyl sulfate. In another aspect, the ASST is a Bacteroides vulgatus ASST. In another aspect, the ASST has at least 90-99% identity to a polypeptide sequence selected from SEQ ID NO: 5-7. In another aspect, the ASST has a polypeptide sequence selected from SEQ ID NO: 5-7.

Another embodiment described herein is a modified Bacteroides vulgatus ASST having at least 90-99% identity to a polypeptide sequence selected from SEQ ID NO: 5-7. In one aspect, the ASST has a polypeptide sequence selected from SEQ ID NO: 5-7. In another aspect, the modified Bacteroides vulgatus ASST further comprising a PelB leader sequence or a signal peptide.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

Mining Annotated Genes for ASSTs from Genome Databases and from Human Fecal Sample Metagenomics Dataset

Genes annotated to encode ASSTs were found via Integrated Microbial Genomes and Microbiomes systems (IMG/M) and NCBI genome annotations by searching with either EC 2.8.2.22 or with the name “aryl-sulfate sulfotransferase” in 135 commonly found human gut microbes. To identify the presence or absence of aryl-sulfate sulfotransferase (asst) genes (KEGG Orthologs) in genomes of bacteria from human fecal samples, we used shotgun metagenomics data and looked for the asst genes in 38 children total (18 children with ASDs and typically developing children). Additional details on the recruitment of participants, sample collection, sequence processing, and data analysis are available in a study by Nirmalkar et al., Int. J. Mol. Sci. 23(21): 13481 (2022). This original study was conducted according to the Declaration of Helsinki and approved by the Arizona State University Institutional Review Board (IRB protocols #00001053 and #00004890). Written informed consent was obtained from all participants' parents or legal guardians. All human samples were de-identified prior to data analysis, and no identifiable information was accessible to the investigators during the current work. Because the present study involved only secondary analysis of previously published fully de-identified data, additional ethical approval was not required. To identify asst genes from bacterial sequences, we used the marker gene-based tool MetaPhlAn4 (Metagenomic Phylogenetic Analysis, v3.1.0) and the taxonomic profiles of bacteria were identified with the ChocoPhlAn pangenome database. Bacterial taxonomic profiles were used with HUMAnN3 (the Human Microbiome Project Unified Metabolic Analysis Network, v3.0 tool with UniRef90 and MetaCyc databases to calculate the bacterial gene abundance for metabolic pathways. Further, gene family abundances from HUMAnN3 were converted to KEGG Orthologs (KOs) to analyze the differential relative abundance of KOs and asst gene.

### Molecular Cloning

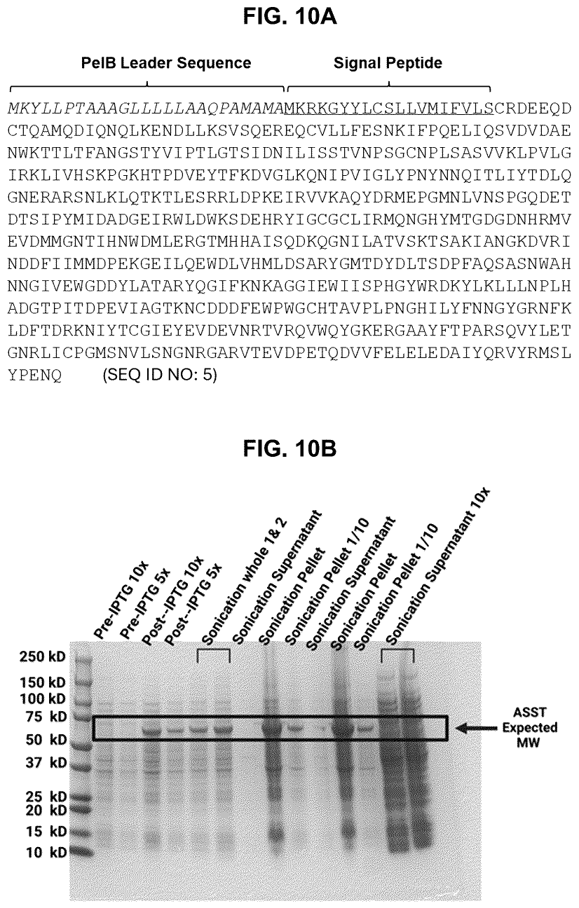

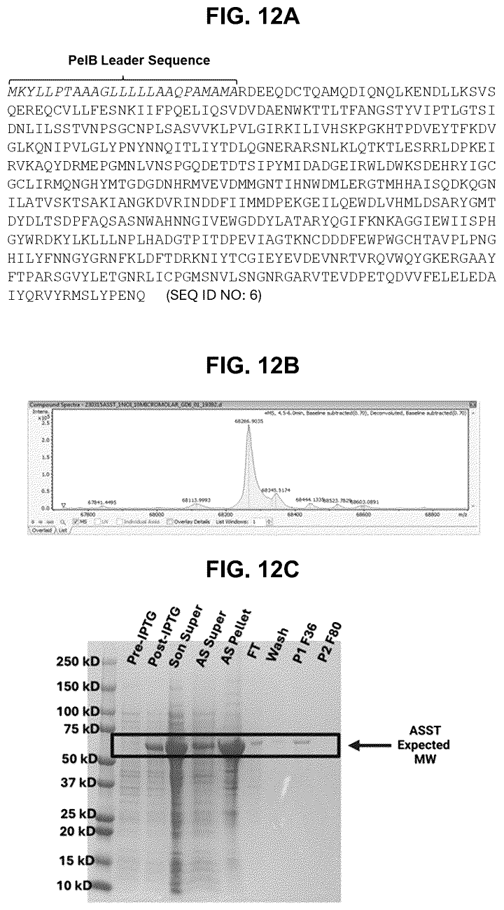

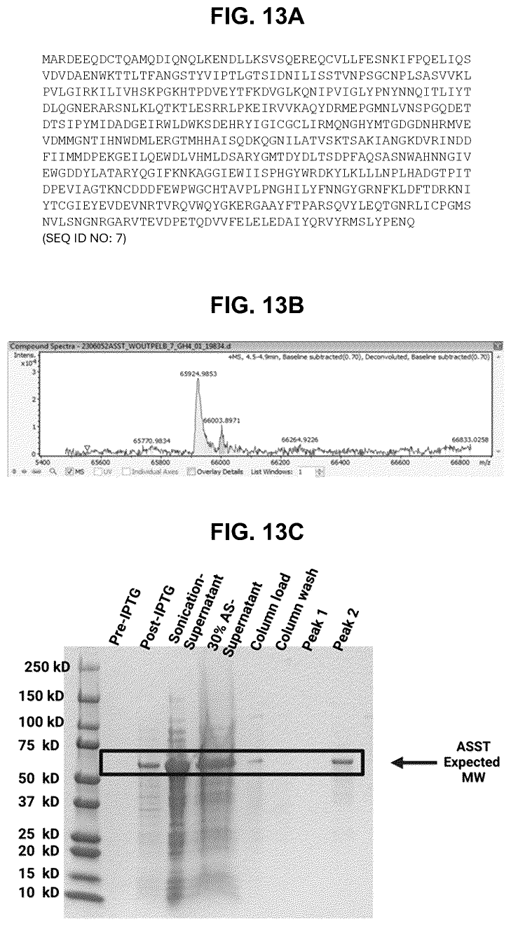

BvASST was identified in preliminary gene product searches using IMG/M database with EC 2.8.2.22. The gene encoding BvASST (Gene ID 640760705; locus tag BVU_RS02960, formerly BVU_0583) (SEQ ID NO: 1) was synthesized and codon-optimized for E. coli expression (Azenta Biosciences) (SEQ ID NO: 3). The gene was cloned into pET-26b using NcoI and SacI to generate pET-26b+BvASST (FIG. 10). A second construct lacking the signal peptide was generated by PCR amplification using the forward primer 5′-CCATGGCCCGCGATGAAGAACAAGATT-3′ (SEQ ID NO: 13) and the reverse primer 5′-GAGCTCTTACTGATTTTCCGGATA-3′ (SEQ ID NO: 14), followed by cloning into pET-26b with NcoI and SacI to yield pET-26b+BvASSTnoSP (FIG. 12-13). Both constructs were transformed into E. coli BL21(DE3) and NEB5α.

### Protein Expression and Purification

Escherichia coli BL21(DE3) cells harboring pET-26b+BvSTnoSP were grown in lysogeny broth (LB) with 0.1 mM kanamycin at 37° C. to an OD₆₀₀ of 0.4 (~3.2×108 bacterial cells/mL). Cultures were then shifted to 20° C. and induced at an OD₆₀₀ of 0.6 (~4.8×108 cells/mL) with 0.1 mM IPTG, followed by overnight expression at 20° C. with shaking at 150 rpm. All the subsequent steps were carried out at 4° C. unless mentioned otherwise. Cells were harvested and resuspended in buffer containing 20 mM Tris, pH 8, supplemented with one cOmplete Mini EDTA-free protease inhibitor tablet per liter of culture, then lysed by sonication (15 cycles of 30 s on/90 s off at 60% duty cycle and tip output of 6 with a Branson Sonifier 250 model 102C [Ce]). Sonicated cells were centrifuged at 18,000×g for 40 min and the supernatant was subjected to 30% (w/v) ammonium sulfate precipitation. Precipitated protein was resuspended in 20 mM Tris, pH 8, filtered through a 0.2-μm polyethersulfone (PES) membrane, diluted to 200 mL, and loaded onto a pre-equilibrated Cytiva HiScreen Capto DEAE column (0.77×10 cm, 4.7 mL) at 1.5 mL/min. After washing with five column volumes of 20 mM Tris containing 50 mM NaCl (pH 8), BvASST was eluted using a linear gradient to 20 mM Tris containing 500 mM NaCl (pH 8) at 1 mL/min. Eluted protein was then buffer exchanged into 20 mM Tris, pH 8 and concentrated with kDa molecular weight cut-off (MWCO) centrifugal filters from Cytiva and stored at −80° C. for enzymatic assays. For mass-spectrometry analysis, the protein was further purified by size-exclusion chromatography on a Cytiva HiPrep 16/60 Sephacyri S-300 (120 mL) column using 20 mM Tris, 50 mM NaCl, pH 8 at 0.5 mL/min. Fractions with BvASST were pooled and concentrated with 30 kDa MWCO spin filters.

### Removal of PelB Leader Sequence

A third construct was created without the PelB leader sequence to check the effect on enzyme activity (Full sequence FIG. 13A; SEQ ID NO: 7). Using restriction cloning, gene encoding ASST was inserted into pET-28a to create the construct pET-28a+BvASST and transformed into E. coli BL21(DE3) for expression and E. coli NEB5a for storage.

Protein Expression and Purification of BvASST without PelB

BvASST protein was purified similar to other constructs with the following modifications: Gene encoding BvASST was cloned into pET-28a for purification. Induction was conducted 37° C. At OD=0.6, cultures were induced with 0.1 mM IPTG. These cultures were allowed to grow for 3 hours at 37° C. with shaking at 150 rpm before harvesting.

### Enzyme Activity Assays

Enzyme activity assays contained 1 mM of sulfo donor, 1 mM of sulfo acceptor, and 4 μM of BvASST. All assays were conducted in 20 mM Tris, pH 8. Absorbance-based assays were performed individually in cuvettes with an Agilent Cary 3500 UV-Vis spectrophotometer and fluorescence-based assays were conducted in black 96-well plates with a Biotek Synergy HTX multimode plater reader in a kinetic mode. Absorbance-based assays utilized p-nitrophenyl sulfate (pNPS) as a sulfo donor and were monitored at 405 nm for the production of p-nitrophenol (pNP), whereas fluorescence-based assays relied on 4-methylumbelliferyl sulfate (4-MUS) as a sulfo donor and were monitored at 360 excitation/460 emission for production of 4-methylumbelliferone (4-MU) using a standard curve. All assays were carried out at 37° C. and at a final volume of 100 μL for absorbance-based assays or 300 μL for fluorescence-based assays. Absorbance-based assays were monitored for 30 min, whereas fluorescence-based assays were monitored for 200 min. All reactions were initiated with the addition of acceptor molecules, which allowed the incubation of enzyme with sulfo group donors. Initial velocities were calculated from the linear portions of the plots of product concentration over time.

Incubation of BvASST with Various Sulfo Group Donors to Detect Sulfonated BvASST

A 50 μM BvASST was incubated with either 0.1 or 1 mM of respective sulfo donor for 20 min at 37° C. in 20 mM Tris, pH 8. After incubation, these samples were spin filtered (30 kDa, MWCO spin filters) and transferred to a final buffer consisting of 5 mM ammonium acetate, pH 7.5 and immediately analyzed via intact mass spectrometry.

Incubation of Sulfonated BvASST with Various Sulfo Group Acceptors to Detect Unsulfonated BvASST

A 50 μM BvASST was incubated with 50 μM pNPS at 37° C. for 20 min in 20 mM Tris, pH 8 to produce sulfonated BvASST. Sulfonated BvASST was then incubated with a variety of sulfo acceptors: p-coumaric acid (pCA), 4-EP, dopamine, pC, acetaminophen, tyramine, and a control without acceptor. Samples were incubated at 37° C. for 2 h in 20 mM Tris, pH 8, before spin filtered (30 kDa, MWCO spin filters) and transferred to a final buffer of 5 mM ammonium acetate, pH 7.5. Control samples were processed similarly but not incubated with any substrate (without donor and acceptor). Samples were kept on ice and immediately analyzed via mass spectrometry. Intact mass analysis was carried out as detailed in the following section.

### Procedure for Electron Transfer Dissociation-Based Site-Specific Determination of Sulfonated Peptides

Final determination of the sulfonated position of the targeted peptide was determined using Electron Transfer Dissociation (ETD) with a supplemental activation of higher-energy collisional dissociation (HCD). The liquid chromatography system was an Ultimate 3000 equipped with a 75 μm×500 mm C18 Easy Spray HPLC column (Thermo Fisher, ES903) with a particle diameter of 2 μm. This was connected to an Orbitrap Fusion Lumos mass spectrometer. The injection protocol for the Ultimate 3000 initially picked up 3 μL of 10% methanol in water that was used as the wash reagent and then discarded. Then 5 μL of water was sampled followed by 1 μL of specimen and then by 2.5 μL of water that was pulled into the 10 μL injection loop. The sample was then directly injected onto the column at a rate of 0.250 μL/min at a mobile phase of 98%/2% (A/B) where A was 0.1% formic Acid in water and B was 0.1% formic acid in acetonitrile. The gradient ramp increased in % B to 10% from the injection time (time zero) until 10 minutes, followed by an immediate ramp to 15% by 15 minutes, then up to 35% by 50 minutes, up to 45% by 60 minutes, and up to 98% by 65 minutes. (There were no pauses between ramps.) The gradient remained steady at 98% B until 85 minutes at which point the gradient decreased to 2.0% B by 90 minutes. The gradient remained steady at 2% until 100 minutes, ending the run.

The Orbitrap resolving power was set to 120 k, with a high mass range and a scan range of 300-3000 m/z units. Maximum injection time was set to 50 ms, with a 100% normalized AGC target with nine minimum points across the peak. All samples were run in the positive ion mode. The targeted sample inclusion list for MS/MS was 1262.8698³⁺ and 1263.5381³⁺; mass tolerance low was 0.5 m/z units and high tolerance was 1.0 m/z units. Data dependent acquisition settings were nine minimum points across the peak using a quadrupole isolation mode and isolation window set to 1.6 m/z units. ETD reaction time was set to 50 ms with a 2×10⁵-reagent target and with a max ETD reagent injection time of 200 ms. Supplemental activation of HCD was set to 20%. The Orbitrap was used as the detector with a resolution of 500K and a AGC target of 5×10⁴, AGC normalization target set to 100% and a maximum injection time of 1014 ms.

### Intact Protein Mass Spectrometry

BvASST spectra were obtained on a Bruker Maxis 4G quadrupole-time of flight instrument with an ESI ion source connected to an Ultimate 3000 capillary HPLC equipped with an autosampler. Samples were diluted with 0.1% (v/v) trifluoroacetic acid to a final concentration of 1 μM, of which 2 μL was injected. Specimens were initially introduced via the loading pump at 10 μL/min with the solvent at an 80/20 mixture of 0.1% formic acid and acetonitrile respectively for 4 min through an Optimize Technologies Protein CapTrap (cat. number 10-04814-TM). After 4 min, a valve switch routed the capillary pump flow over the CapTrap, carrying the protein from the CapTrap into the mass spectrometer at 3 μL/min with a mixture of 0.1% formic acid (solvent A) and 100% acetonitrile (solvent B) at a composition of 65/35% (A/B), which was held until 4.6 min when the stepwise gradient was changed to 55/45% (A/B) over 0.1 min. At 6.6 min, the solvent changed to 35/65% (A/B) over 0.1 min, which then ran until 7.5 min when the solvent composition was changed to 20/80% (A/B) over 0.1 min. Finally, at 9.6 min, the solvent was returned to the baseline of 65/35% (A/B) and remained that way for the rest of the 10-min run. The Bruker Maxis 4G ESI-Q-TOF was set in positive ion mode over an m/z range of 300-3,000. The potential on the inlet capillary was set to 4,000 V and the end plate offset was 500 V. Nebulizer nitrogen was 3 bar and the dry gas nitrogen was set to 4.0 L/min at 225° C. The sampler rate of the digitizer was 4 GHz with a spectra rate summation of 1 Hz. Data analysis was performed using Bruker Daltronic Compass Software wherein spectra were averaged from 4.6 to 7 min. Baseline subtraction of this summation was done at 0.7 flatness, and then subsequently the raw mass spectra were charge-deconvoluted. Following deconvolution another baseline subtraction was performed to visualize peaks for analysis.

### Trypsin Digestions and Mass Spectrometry Visualization

Reduction, alkylation and tryptic digestion of BvASST was performed using the S-Trap Micro-Spin Column protocol obtained from the manufacturer (Protifi, C02-micro-10). In brief, BvASST specimens were diluted to a concentration of 7 μg/μL in mass spectrometry grade water (Fisher Chemical, W6-4). 10 μL of the diluted protein solution was mixed with 10 μL of lysis buffer consisting of 10% sodium dodecyl sulfate (SDS) (Sigma-Aldrich, 436143) and 100 mM triethylammonium bicarbonate (TEAB) (Thermo Fisher, P190114), followed by the addition of 1 μL of 1 M dithiothreitol (DTT) creating a final concentration of 50 mM DTT (Sigma-Aldrich, Cat. 10708984001). Samples were then incubated for 20 minutes at 80° C. Next, 1 μL of 1 M iodoacetamide (IAA) (G-Biosciences, 786-228) was added to bring the final IAA concentration to 45 mM, followed to an additional 30-min incubation in the dark. 1.4 μl of 85% phosphoric acid (Fluka, Cat. 79600) was added to the 22 μL of specimen to create a final concentration of 5% phosphoric acid. A trypsin stock solution (at 1 μg/μL) was prepared by reconstituting 20 μg of sequencing grade modified trypsin (Promega, V5111) in 20 μL of 50 mM acetic acid. 2 μL of this solution (containing 2 μg of trypsin) was added to samples and mixed immediately and thoroughly; subsequently 25.4 μL of samples were added to the S-Trap and mixed with 167 μL of binding buffer consisting of 90% aqueous methanol (ACS, BDH1135) and 100 mM TEAB at a pH of 7.1. The mixture was then spun in a centrifuge at 4000×g for one minute and the process was repeated four times by adding 150 μL of the binding buffer each time. Wash throughs were discarded. Then 0.5 μL of the 1 μg/μL trypsin stock was added to 24.5 μL of 50 mM TEAB (pH 8) which was then added to the top of the S-Trap. Then the S-Trap was incubated at 47° C. for 1.5 hours while loosely capped and in contact with a heating block. Following incubation, peptides were eluted into a new clean 1.5 mL aliquot tube, by adding 40 μL of 50 mM TEAB and then spinning for 1 min at 4000×g, collecting the eluent into a new 1.5 mL Eppendorf tube. Next, 40 μL of 0.2% formic acid (Fisher Chemical, CAS 64-18-6) was added and spun for 1 min at 4000×g, collecting the eluent into a new 1.5 mL Eppendorf tube. Finally, 35 μL consisting of 50% acetonitrile (Fisher Chemical, CAS A955-4) with 0.2% formic acid was added to the S-Trap and spun for 1 min at 4000×g, collecting the eluent into a new 1.5 mL Eppendorf tube. All elutions were combined and dried down in a speed vac for two hours and subsequently resuspended in 30 μL of 0.1% trifluoroacetic acid (TFA) (TCI, T0431) and frozen at −80° C. until analysis.

### Sample Preparation for Anion Analysis

For IC analysis of sulfated compounds, compounds were incubated in 20 mM Tris, pH 8 at concentrations of either 500 μM or 1 mM depending on compound availability. Incubation lasted 4 hours at 37° C. to simulate extensive reaction conditions then analyzed immediately via ion chromatography (IC). Percent sulfate lost was calculated comparing the free sulfated measured in solution compared to known initial concentration of substrate added. For the preparation of sulfated enzyme 10 μM of BvASST was incubated with 1 mM pNPS for 20 minutes at 37° C. before spin filtering (5 kDa) to remove remaining pNPS. Enzyme was resuspended in 20 mM Tris, pH 8 and incubated at 37° C. for 4 hours before spin filtering again (5 kDa) to remove protein. Supernatant was analyzed via IC immediately.

### Anion Analysis by Ion Chromatography (IC)

The anion analysis was performed using a Compact IC Flex by Metrohm (Model 930) and IC Sample Center by Metrohm (Model 889). The IC system was equipped with a Metrosep A Supp 7—150/4.0 column (Model 6.1006.620) and Metrohm A Supp 5 Guard/4.0 guard column. The mobile phase consisted of 3.6 mM sodium carbonate (Na₂CO₃), 4 mM sodium bicarbonate (NaHCO₃), 8% (v/v) acetone, 5% (v/v) methanol, DI water (18 ohm), and it was run at a flow rate of 0.8 mL/min. The column temperature was set and maintained at 55° C. throughout the analysis. The injection volume used for each sample was 100 μL.

### Chromatographic Peaks Identification and Quantification

Chromatographic peaks were identified by using MagIC Net 3.3 software and by comparing their retention times with those of the standard anions. The standards used for this analysis were high purity IC standards. These standards consist of 600 μg/mL phosphate (PO₄³⁻), 400 μg/mL bromide (Br—), 400 μg/mL nitrate (NO₃⁻), 400 μg/mL sulfate (SO₄²⁻), 200 μg/mL chloride (Cl⁻), and 100 μg/mL fluoride (F⁻). Each sample was run and analyzed in triplicate, and the results were averaged to determine the final concentration of each anion.

### Initial Analysis of BvASST Peptides by Collision Inducted Dissociation-Based LC-MS/MS

Initial analysis by LC-MS/MS was performed using an Ultimate 3000 HPLC equipped with an Optimize Technologies peptide captrap (10-04813-TN) and a BIOshell™ A160 Peptide C18 column 150 mm×0.2 mm (Sigma-Aldrich, 67206-u), connected to a Bruker Maxis 4G quadrupole time of flight instrument with an electrospray ionization (ESI) source. 1 μL of reconstituted tryptic digest products was diluted with 29 μL of 0.1% TFA. Subsequently, 2 μL of this dilution was injected onto the captrap. Major Bruker Maxis 4G settings were as follows: End plate offset to 500 V, Capillary to 4000 V, Nebulizer to 3.0 bar, dry gas 4.0 L/min at 225° C. and mass range was set to 200-2500 m/z. Sampler rate of digitizer was 4 GHz with a spectra rate summation of 1 Hz.

Chromatography protocol was as follows: 2 μL of the tryptic digests were injected and loaded onto the captrap at 10 μL/min using 98%/2% (A/B) mobile phase where A represents a mixture of 80/20% mixture of 0.1% formic acid (80%) and acetonitrile (20%) and B represents 100% acetonitrile. Loading continued for a total of four minutes before a 10-port valve switch routed the capillary pump flow through the captrap into the column at a rate of 2 μL/min and an initial composition of 98% of 0.1% formic acid in water (A) and 2% of acetonitrile (B). Starting at 4.5 minutes % B was raised linearly at a rate of 2.6%/min until a final composition of 80% B was reached at 34.5 minutes. At 34.5 minutes to 35.5 minutes % B was increased to 95% at a rate of 15%/min and was held at 95% B until 40.5 minutes. At 40.5 minutes to 42.5 minutes % B was decreased to 2% at a rate of 46.5%/min and held there until 52.5 minutes, ending the run.

For MS/MS analysis of sulfonated peptide fragments, a scheduled precursor list was utilized for the m/z region from 1263.5 to 1263.90 with a single mass isolation width of 10 m/z units. Auto MS/MS was utilized with a time slice of 2 seconds, three precursors, and a threshold of 200 counts. Collision energy for auto MS/MS was set to scale based on precursor ion mass and charge state. For the triply charged precursor at m/z 1263, the collision energy was 37.5 eV.

### Collision-Induced Dissociation (CID)-Based LC-MS/MS

The trypsin-digested products of a 70-μg BvASST sample were separated on an Ultimate 3000 liquid chromatography system equipped with a BIOshell A160 Peptide C18 column (150 mm×0.2 mm, 2.7 μm particle diameter size; Sigma-Aldrich, 67206-u) and eluted into a Bruker Maxis 4G quadrupole-time-of-flight mass spectrometer. Auto MS/MS was performed using a scheduled protocol list for 1,263.5 to 1,263.90 with CID energy set to scale between 30 and 50 eV, based on charge state.

### Electron-Transfer/Higher-Energy Collision Dissociation (EThcD)-Based LC-MS/MS

Peptides from trypsin-digested BvASST were injected into an Orbitrap Fusion Lumos through an Ultimate 3000 using a C18 Easy Spray HPLC column (75 μm×500 mm, 2 μm particle size; Thermo Fisher, ES903). Analysis by MS/MS was done using a target inclusion list for 1,262.8698³⁺ and 1,263.5381³⁺ with electron transfer dissociation set at 50 ms reagent time and a supplemental activation for higher-energy collisional dissociation (HCD) set at 20%. (detailed in Supplementary Methods). Fragmentation carried out in this way tends to produce c and z ions in addition to b and y ions that are commonly produced by conventional CID.

### Mass Spectrometry Data Processing

Theoretical fragment masses were calculated using Protein Prospector. Carbamidomethylation of cysteine was set as a fixed modification to account for alkylation by iodoacetamide. Sulfohistidine was set as a fixed modification on His471 using the User-Specified Elemental Composition feature to account for the 80 Da mass shift associated with sulfonation. Data from Orbitrap EThcD runs were analyzed using the FreeStyle Software from Thermo Fisher Scientific. MS/MS scans were averaged over the chromatographic elution profile of m/z 1,263.5670³⁺ to generate an information-rich MS/MS spectrum. Masses of observed monoisotopic peaks from fragment ions were compared against calculated fragment masses to calculate delta and ppm values. Assignments were made for observed fragments within ±0.007 m/z units or ±10 ppm from their theoretical (calculated) m/z values and documented in Table 1, as well as in the MS/MS spectrum used to make those assignments.

TABLE 1
EThcD MS/MS Analysis of a Sulfonated Peptide

| Observed | | Presumptive | Calculated | Delta Value | |
|---|---|---|---|---|---|
| m/z | Charge | ID | m/z | (m/z) | ppm |
| 217.1309 | 1+ | (z + 1)₂ | 217.1295 | 0.0014 | 6.2403 |
| 232.1418 | 1+ | y₂ | 232.1404 | 0.0014 | 6.1800 |
| 380.1953 | 1+ | (z + 1)₃ | 380.1928 | 0.0025 | 6.6001 |
| 395.2062 | 1+ | y₃ | 395.2037 | 0.0025 | 6.2893 |
| 410.1769 | 1+ | Unassigned | | | |
| 437.2171 | 1+ | (z + 1)₄ | 437.2143 | 0.0028 | 6.2908 |
| 452.2280 | 1+ | y₄ | 452.2252 | 0.0028 | 6.2148 |
| 494.2387 | 1+ | Unassigned | | | |
| 551.2606 | 1+ | (z + 1)₅ | 551.2572 | 0.0034 | 6.1636 |
| 566.2716 | 1+ | y₅ | 566.2681 | 0.0035 | 6.1779 |
| 665.3041 | 1+ | (z + 1)₆ | 665.3002 | 0.0039 | 5.8258 |
| 680.3148 | 1+ | y₆ | 680.3111 | 0.0037 | 5.4381 |
| 781.2499 | 1+ | b₆ | 781.2457 | 0.0042 | 5.3640 |
| 798.2764 | 1+ | c₆ | 798.2723 | 0.0041 | 5.1767 |
| 811.3650 | 1+ | z₇ | 811.3608 | 0.0042 | 5.2211 |
| 827.3841 | 1+ | y₇ | 827.3795 | 0.0046 | 5.5887 |
| 916.4707 | 2+ | y₁₆⁺² | 916.4656 | 0.0051 | 5.6069 |
| 967.3293 | 1+ | b₇ | 967.3251 | 0.0042 | 4.3610 |
| 974.4287 | 1+ | z₈ | 974.4241 | 0.0046 | 4.7178 |
| 1081.4090 | 1+ | c₈ | 1081.4044 | 0.0046 | 4.2697 |
| 1087.5132 | 1+ | z₉ | 1087.5081 | 0.0051 | 4.6857 |
| 1103.5313 | 1+ | y₉ | 1103.5269 | 0.0044 | 3.9521 |
| 1148.9728 | 2+ | b₁₉⁺² | 1148.9806 | −0.0078 | 6.8173 |
| | | (No sulf) | | | |
| 1200.5974 | 1+ | z₁₀ | 1200.5922 | 0.0052 | 4.3047 |
| 1221.5469 | 3+ | MH-44.0374 | 1221.5384 | 0.0085 | 6.9494 |
| | | (No Sulf) | | | |
| 1230.2191 | 3+ | MH-18.0106⁺³ | 1230.214 | 0.0051 | 4.1246 |
| | | (No Sulf) | | | |
| 1236.2246 | 3+ | MH⁺³ | 1236.2175 | 0.0071 | 5.7636 |
| | | (No Sulf) | | | |
| 1262.8763 | 3+ | Precursor | 1262.8698 | 0.0065 | 5.1645 |
| 1267.4891 | 1+ | c₉ | 1267.4837 | 0.0054 | 4.2868 |
| 1324.5109 | 1+ | c₁₀ | 1324.5051 | 0.0058 | 4.3657 |
| 1337.6573 | 1+ | z₁₁ | 1337.6511 | 0.0062 | 4.6401 |
| 1353.6743 | 1+ | y₁₁ | 1353.6698 | 0.0045 | 3.3366 |
| 1370.6749 | 2+ | y₂₄⁺² | 1370.6637 | 0.0112 | 8.1672 |
| | | (No Sulfo) | | | |
| 1394.6781 | 1+ | z₁₂ | 1394.6726 | 0.0055 | 3.9721 |
| 1410.6968 | 1+ | y₁₂ | 1410.6913 | 0.0055 | 3.8762 |
| 1452.7076 | | Unassigned | | | |
| 1463.7166 | 2+ | y₂₅⁺² | 1463.7034 | 0.0132 | 9.0493 |
| | | (No Sulfo) | | | |
| 1484.5409 | 1+ | c₁₁ | 1484.5358 | 0.0051 | 3.4584 |
| 1495.6773 | 2+ | z₂₅⁺² | 1495.6724 | 0.0049 | 3.3003 |
| 1508.7201 | 1+ | z₁₃ | 1508.7155 | 0.0046 | 3.0301 |
| 1560.6925 | 2+ | z₂₆⁺² | 1560.6976 | −0.0051 | 3.2866 |
| 1593.7556 | 2+ | z₂₇⁺² | 1593.7495 | 0.0061 | 3.8145 |
| | | (No Sulfo) | | | |
| 1601.7630 | 2+ | y₂₇⁺² | 1601.7589 | 0.0041 | 2.5297 |
| | | (No Sulfo) | | | |
| 1620.5927 | 2+ | Unassigned | | | |
| 1628.7125 | 2+ | Unassigned | | | |
| 1633.7350 | 2+ | z₂₇⁺² | 1633.7279 | 0.0071 | 4.3336 |
| 1641.7355 | 2+ | y₂₇⁺² | 1641.7373 | −0.0018 | 1.1177 |
| 1651.2619 | 2+ | z₂₈⁺² | 1651.263 | −0.0011 | 0.6387 |
| | | (No Sulfo) | | | |
| 1659.2694 | 2+ | y₂₈⁺² | 1659.2723 | −0.0029 | 1.7546 |
| | | (No Sulfo) | | | |
| 1668.7743 | 2+ | Unassigned | | | |

Fragment ion assignments from EThcD with observed b/c/y/z ions showing retention or loss of the sulfo group.

### HPLC Analysis of BvASST Catalyzed Reactions

To detect production of sulfated acceptor molecules, all enzymatic reactions were analyzed via HPLC with the Waters e2695 separation module using a Synergi 4 μm Hydro-RP 80 A 250×4.6 mm column, under gradient conditions using buffer A containing methanol with 0.1% formic acid and buffer B containing water with 0.1% formic acid. The optimized gradient method started with 5% A: 95% B for 0-5 min, to 20% A: 80% B from 5 to 19 min, this changed to 80% A: 20% B from 19 to 28 min. This combination was held from 28 to 35 min. After the hold for 7 min, the mixture was changed to 5% A: 95% B from 35 to 37 min, which was then converted to starting condition of 5% A: 95% B from 37 to 50 min at a flow rate of 1 mL/min. All samples were monitored by a photodiode array detector. Specific wavelengths used for data analysis were 243 nm, 265, 276, and 315 nm. All enzymatic reactions and controls (n=3) were filtered through 10 kDa MWCO spin filters and stored at −80° C. before being analyzed by HPLC. All products from enzyme catalysis were identified by comparing their retention times to authenticated standards purchased commercially.

### Enzyme Concentration Variation Experiments

Enzyme activity assays were conducted with various acceptors and pNPS as a donor. For each reaction using a unique acceptor and the donor pNPS, BvASST concentrations were varied from 0 to 8 μM in 2 μM increments. Concentrations of pNPS and acceptor were held at 1 mM. Reactions were carried out in 96-well plates in 20 mM Tris, pH 8 to a final volume of 300 μL for 150 min at 37° C. Reactions were initiated by adding acceptor molecules. Initial velocities were determined from the linear portion of the progress curves as μM pNP formed per minute.

### Steady-State Kinetics

Steady-state kinetic parameters were measured with activity assays by varying either donor or acceptor concentrations.

Varying Acceptor Concentrations with a Fixed Concentration of pNPS as a Donor

Rates of BvASST-catalyzed reactions were measured with 1 mM pNPS as a sulfo donor and varying concentrations of acceptor substrates, including pCA (0.039 μM to 2.25 mM), dopamine (0.198 μM to 3.38 mM), acetaminophen (0.198 to 10 mM), 4-EP (0.250 to 5 mM), pC (25 μM to 1.6 mM), and caffeic acid (12.5 to 500 μM). Substrate concentration ranges were selected to achieve saturation for each acceptor. Reactions were carried out in 20 mM Tris, pH 8 with 4 μM of BvASST at 37° C. for 30 min and monitored by measuring pNP formation at 405 nm. Reactions were initiated by addition of the acceptor after allowing the donor and enzyme to equilibrate. Initial velocities (V0) were obtained by fitting the initial time point data to linear regression. These calculated initial rates were then plotted against the concentrations of each acceptor molecule. Kinetic parameters were determined by fitting the resulting datasets to a hyperbolic model. For volatile acceptors (pC, 4-EP), reactions were conducted similarly in covered clear 96-well plates.

Varying Acceptor Concentrations with a Fixed Concentration of 4-MUS as a Donor.

Rates of BvASST-catalyzed reactions were measured with 1 mM 4-MUS as a sulfo donor and varying concentrations of acceptor substrates, including pCA (50 μM to 1.5 mM), dopamine (62.5 μM to 1 mM), acetaminophen (125 μM to 2 mM), 4-EP (125 μM-4 mM), pC (125 μM-4 mM), and caffeic acid (3.1-200 μM). Different concentration ranges were used for each molecule based on the concentration needed to saturate the enzyme velocity. Reactions were carried out in 20 mM Tris, pH 8 with 4 μM of BvASST at 37° C. for 200 min and monitored by measuring the fluorescence of the product, 4-MU (360 excitation/460 emission) in black 96-well plates. Kinetic parameters were determined as described above for assays conducted with pNPS as the sulfo donor. Reactions using either pNPS or 4-MUS as the donor were started with the addition of acceptor molecule after allowing the donor and enzyme to equilibrate. Steady-state kinetics data were fitted with Michaelis-Menten equation (Eq. 1).

$${V}_{0} = {V}_{m ax} \frac{[ S ]}{{K}_{M}} + [ S ] \qquad ( 1 )$$

### Putative Genes Annotated to Encode Microbial ASSTs are Prevalent in Gut Microbes

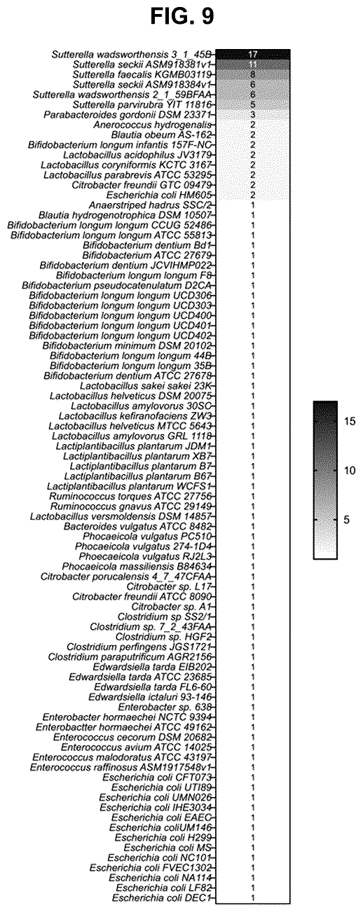

To explore the possibility of sulfonation by microbial ASSTs being part of gut microbiota transformations, we investigated the genomes of commonly found gut microbes. As seen in FIG. 9, various gut bacterial strains, spanning multiple genera known to inhabit human gastrointestinal tract, had genes annotated to encode ASSTs. Many displayed multiple putative asst genes within their genomes that were not direct copies. Particularly, microbes of the genus Sutterella displayed anywhere from two to 17 asst genes. Separate from available genome databases, we identified putative genes annotated as ASSTs in a metagenome dataset from human fecal samples, where we found the presence of asst genes in eight out of 38 samples (Table 2). This analysis was not to compare abundances but to inquire about the presence of these genes in fecal samples. Although these genes have not been highly studied and hence might be lacking in many databases, we were able to confirm their presence in ~20% of our previously sequenced samples.

TABLE 2
Metagenomics Data from Human Fecal Samples

| | | K01023: arylsulfate | |
|---|---|---|---|
| | | sulfotransferase (ASST), | |
| | | EC: 2.8.2.22 relative | ASST Gene: |
| Sample ID | Group | abundance | Presence/Absence |
| MS124 | ASD_Baseline | 0 | No |
| MS125 | ASD_Baseline | 0 | No |
| MS126 | ASD_Baseline | 3.09 × 10⁻⁷ | Yes |
| MS127 | ASD_Baseline | 0 | No |
| MS128 | ASD_Baseline | 0 | No |
| MS129 | ASD_Baseline | 0 | No |
| MS130 | ASD_Baseline | 6.79 × 10⁻⁸ | Yes |
| MS131 | ASD_Baseline | 0 | No |
| MS132 | ASD_Baseline | 1.10 × 10⁻⁷ | Yes |
| MS133 | ASD_Baseline | 0 | No |
| MS134 | ASD_Baseline | 0 | No |
| MS135 | ASD_Baseline | 0 | No |
| MS136 | ASD_Baseline | 0 | No |
| MS137 | ASD_Baseline | 0 | No |
| MS138 | ASD_Baseline | 0 | No |
| MS139 | ASD_Baseline | 0 | No |
| MS140 | ASD_Baseline | 1.13 × 10⁻⁷ | Yes |
| MS141 | ASD_Baseline | 0 | No |
| MS160 | TD/Controls | 0 | No |
| MS161 | TD/Controls | 0 | No |
| MS162 | TD/Controls | 4.37 × 10⁻⁷ | Yes |
| MS163 | TD/Controls | 0 | No |
| MS164 | TD/Controls | 1.61 × 10⁻⁷ | Yes |
| MS165 | TD/Controls | 0 | No |
| MS166 | TD/Controls | 0 | No |
| MS167 | TD/Controls | 0 | No |
| MS168 | TD/Controls | 0 | No |
| MS169 | TD/Controls | 0 | No |
| MS170 | TD/Controls | 1.80 × 10⁻⁷ | Yes |
| MS171 | TD/Controls | 0 | No |
| MS172 | TD/Controls | 0 | No |
| MS173 | TD/Controls | 0 | No |
| MS174 | TD/Controls | 0 | No |
| MS175 | TD/Controls | 0 | No |
| MS176 | TD/Controls | 3.34 × 10⁻⁷ | Yes |
| MS177 | TD/Controls | 0 | No |
| MS178 | TD/Controls | 0 | No |
| MS179 | TD/Controls | 0 | No |

BvASST is Only Soluble without Lipoprotein Signal Peptide

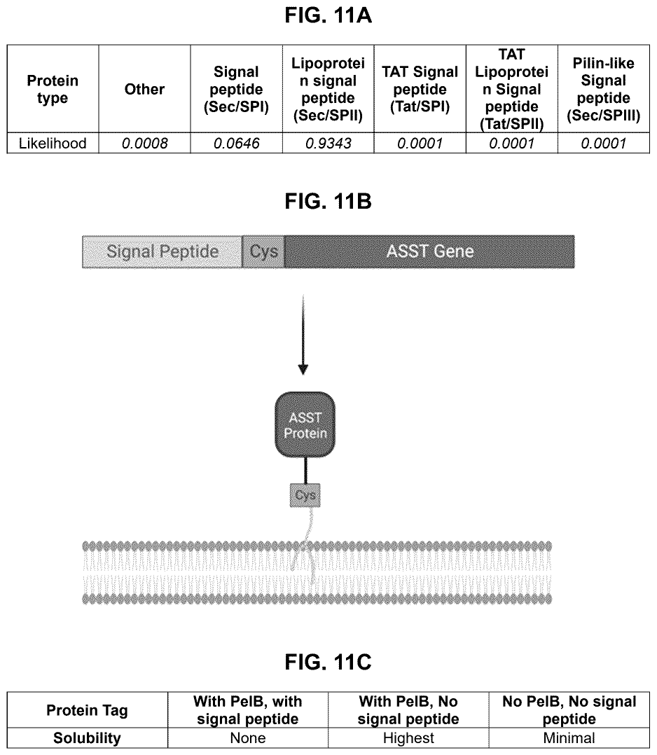

Our initial attempts to purify aryl-sulfate sulfotransferase (BvASST) from Bacteroides vulgatus resulted in insoluble protein aggregates (FIG. 10B). The induced protein was found in the sonication pellet fraction where cell debris are present and isolated from free floating proteins. Following this, we used SignalP 6.0 to determine if a signal peptide was present on BvASST. The program predicted that the enzyme contained a lipoprotein signal peptide (SEC/SPII) with a probability of 0.93 (FIG. 11A). Extensive studies of lipoproteins in bacteria have determined a characteristic cysteine residue at the N-terminus where lipids can be tethered and subsequently get embedded into the membrane with a lipid anchor from this site. Such lipoproteins can be translocated either to the periplasmic leaflet or to outer membrane. To obtain soluble protein, we removed the signal peptide of BvASST. The gene without the signal peptide was then cloned with a N-terminus pelB leader sequence. The pelB signal peptide is expected to direct the protein into the periplasm via the SEC pathway. With the lipoprotein signal peptide removed and an added pelB, BvASST was untethered from the membrane and was purified as a soluble form (FIG. 12C).

However, upon obtaining intact mass spectra of the purified BvASST (FIG. 12B), we observed that the pelB signal peptide was not being cleaved. In E. coli, the signal peptide or pelB is typically cleaved by signal peptidases, which was not observed in our construct. We further confirmed this via sequencing our entire vector that contained the gene of interest and found an intact signal peptidase cleavage site. This lack of cleavage has been seen before and is hypothesized due to the inability of the periplasmic peptidases to access the cleavage site. However, currently the localization of BvASST in both E. coli and B. vulgatus is unknown. We made a construct of BvASST without pelB (FIG. 13). Our attempts to purify BvASST without the pelB tag resulted in very low yields. The average protein yield after elution from the anion exchange column, was 0.34 mg/L, much lower compared to the yield obtained from the protein with pelB tag, which was 10 mg/L (FIG. 11C).

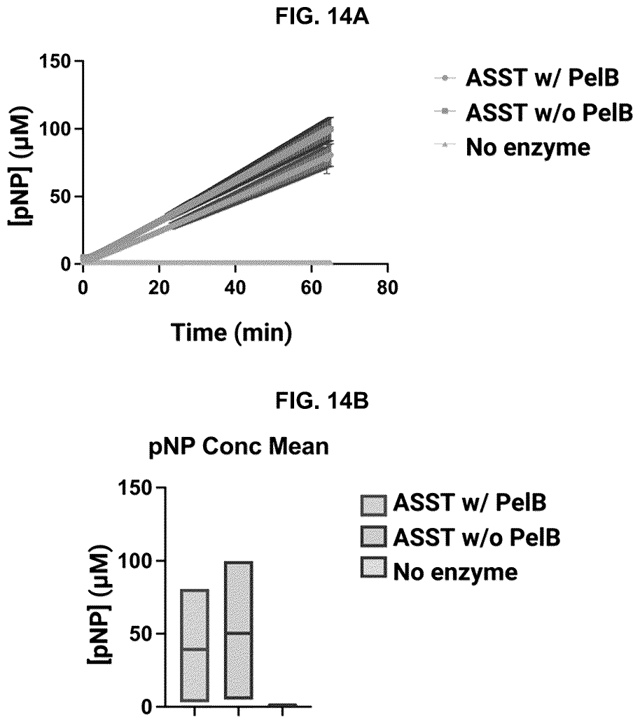

Moreover, we performed activity assays to assess if the addition of pelB tag had any effect on the protein's catalytic ability. As can be seen in FIG. 14, no significant difference in activity was seen for BvASST with or without pelB. For all subsequent assays, BvASST with the pelB tag was utilized.

The Sulfonation State of BvASST in the Presence of Various Phenolic Molecules, Both Sulfated and Nonsulfated, Reveals the Identities of Sulfo Group Donors and Acceptors used by this Enzyme

Despite the high diversity of gut microbial asst genes, for this study, we chose to focus on an annotated asst gene from B. vulgatus ATCC 8482 (now P. vulgatus) for several reasons: (i) the prevalence of Bacteroides in the human gut microbiome, (ii) the presence of a unique lipoprotein signal peptide identified in the translated protein through in silico analysis, (iii) existing studies on B. thetaiotaomicron that describe a different class of sulfotransferases, and (iv) the reduced complexity in selecting an organism with only one annotated asst gene, facilitating future mechanistic studies. Our initial attempts to purify ASST (BvASST) from B. vulgatus resulted in insoluble protein aggregates (FIG. 10B). Following this, we used SignalP 6.0 to determine whether a signal peptide was present on BvASST. The program predicted that the enzyme contained a lipoprotein signal peptide (SEC/SPII) with a probability of 0.93 (FIG. 11A). With the removal of lipoprotein signal peptide and an addition of pelB leader sequence, BvASST was potentially untethered from the membrane and was purified in a soluble form (FIG. 12) when expressed in E. coli. This form of the enzyme was used in all subsequent experiments, particularly because the presence of the pelB sequence had little to no impact on BvASST activity (FIG. 13-14). In addition, we evaluated BvASST activity across a range of pH values and observed slightly higher activity at pH 9. However, because some substrates used in this study are unstable at pH 9, all assays were conducted at pH 8.

All previous studies characterizing ASSTs have been largely confined to utilizing synthetic molecules like pNPS and 4-MUS as sulfo donors, which are not found in physiological conditions. Other than these synthetic molecules, the identity of sulfo donors for ASSTs continues to remain elusive, whereas mammalian sulfotransferases are known for their stringent specificity to use 3′-phosphate-5′-phosphosulfate (PAPS) as a sulfo group donor. To identify potential sulfo donors with known physiological relevance to human health, we leveraged the catalytic mechanism that is characteristic of this class of enzymes known as ping-pong bi-bi. Within this mechanism, the two substrates, identified as the donor of the sulfo group and the acceptor of the sulfo group, interact with the enzyme in separate and successive stages rather than concurrently (FIG. 2). Initially, the sulfo group donor binds to the enzyme's active site and transfers its sulfo group to the enzyme, departing in a desulfated form. This first half reaction temporarily converts the enzyme into a sulfonated intermediate (FIG. 2). In the second half reaction, the sulfonated enzyme intermediate can now bind and transfer a sulfo group to an acceptor molecule to generate sulfated acceptor. During this process, the enzyme reverts to its original unsulfonated form that is ready to start another catalytic cycle.

Taking advantage of this reaction mechanism, we utilized the intact protein mass-spectrometry-based method described by Malojcic et al. and developed it into a screening assay to identify phenolic sulfo group donors and acceptors for BvASST. Malojcic et al., Proc. Natl. Acad. Sci. USA 105(49): 19217-19222 (2008).

Initially, we measured the intact molecular mass of the protein with and without incubation with various sulfo group donor molecules (Tables 3-4). The unsulfonated form of the enzyme has a molecular mass of 68,267 Da, and when the enzyme adopts an intermediate sulfonated form, a mass shift of +80 Da is expected. To screen sulfo group donors, BvASST was incubated with various phenolic sulfate molecules encountered by humans. Additionally, due to the predicted reversibility of ASST-catalyzed reactions, a sulfated acceptor molecule produced by this reaction has the potential to act as a sulfo donor in the reverse direction. With this rationale, we tested commercially available sulfated forms of previously characterized acceptors known to interact with other microbial ASSTs. These molecules include pCA sulfate (pCAS), acetaminophen sulfate (acetS), indoxyl sulfate (indoxylS), pC sulfate (pCS), dopamine sulfate (dopamineS), serotonin sulfate (serotoninS), and PAPS. In addition, we incubated BvASST with synthetic sulfo donors like pNPS and 4-MUS.

TABLE 3
Intact MS Peak Intensity Values

| | | Non- | | |
|---|---|---|---|---|
| | Sulfonated | Sulfonated | | Non- |
| | Peak | Peak | Sulfonated | Sulfonated |
| Compound | Intensity | Intensity | (%) | (%) |
| pNPS | 94293 | 3378 | 96.54 | 3.46 |
| pCAS | 50882 | 1149 | 97.79 | 2.21 |
| acetaminophen | 28779 | 35988 | 44.43 | 55.57 |
| sulfate | | | | |
| indoxyl sulfate | 15254 | 40521 | 27.35 | 72.65 |
| dopamine | 15837 | 53963 | 22.69 | 77.31 |
| sulfate | | | | |
| serotonin sulfate | 15114 | 50116 | 23.17 | 76.83 |
| 4-MUS | 105501 | N/A | 100 | 0 |
| PAPS | 4823 | 47647 | 9.19 | 90.81 |
| No substrate | 5536 | 29399 | 15.85 | 84.15 |

BvASST incubated with 100 μM of each donor. Intensities for sulfated (m/z = M + 81) and non-sulfated (m/z = M + 1) peaks.

TABLE 4
Intact MS Peak Intensity Values

| | | Non- | | |
|---|---|---|---|---|
| | Sulfonated | Sulfonated | | Non- |
| | Peak | Peak | Sulfonated | Sulfonated |
| Compound | Intensity | Intensity | (%) | (%) |
| pNPS | 43519 | 1548 | 96.57 | 3.43 |
| pCAS | 57976 | N/A | 100.00 | 0.00 |
| acetaminophen | 79691 | 5607 | 93.43 | 6.57 |
| sulfate | | | | |
| indoxyl sulfate | 11198 | 3615 | 75.60 | 24.40 |
| dopamine sulfate | 35474 | 14580 | 70.87 | 29.13 |
| serotonin sulfate | 37524 | 35206 | 51.59 | 48.41 |
| 4-MUS | 90130 | N/A | 100 | 0 |
| PAPS | 15997 | 109935 | 12.70 | 87.30 |
| No substrate | 5536 | 29399 | 15.85 | 84.15 |

BvASST incubated with 1 mM of each donor. Intensities for sulfated (m/z = M + 81) and non-sulfated (m/z = M + 1) peaks.

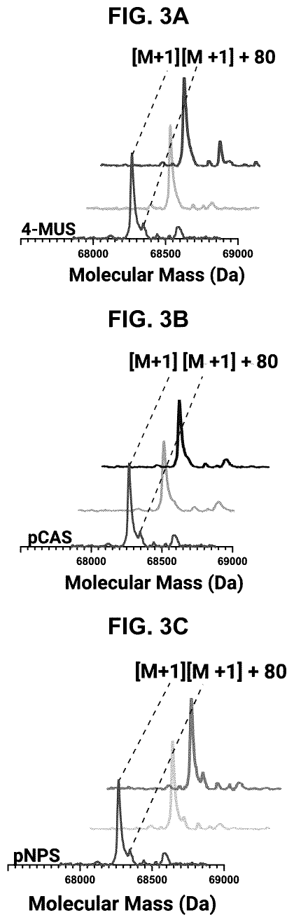

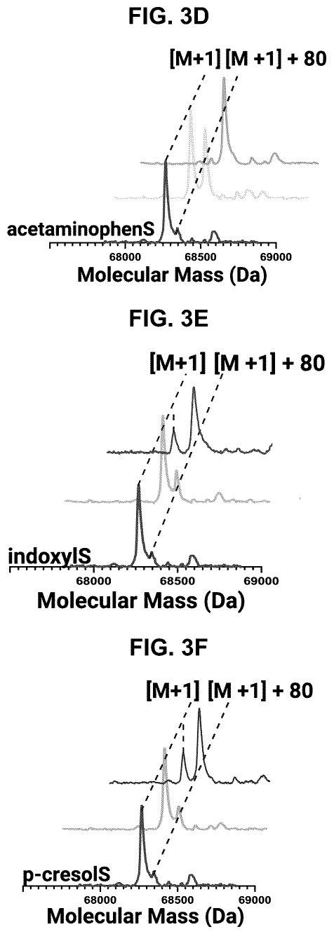

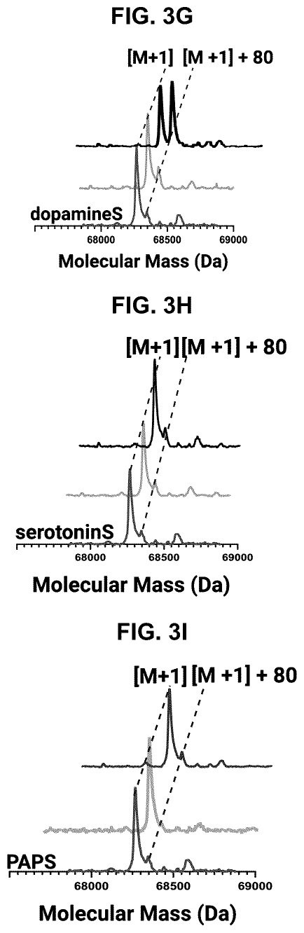

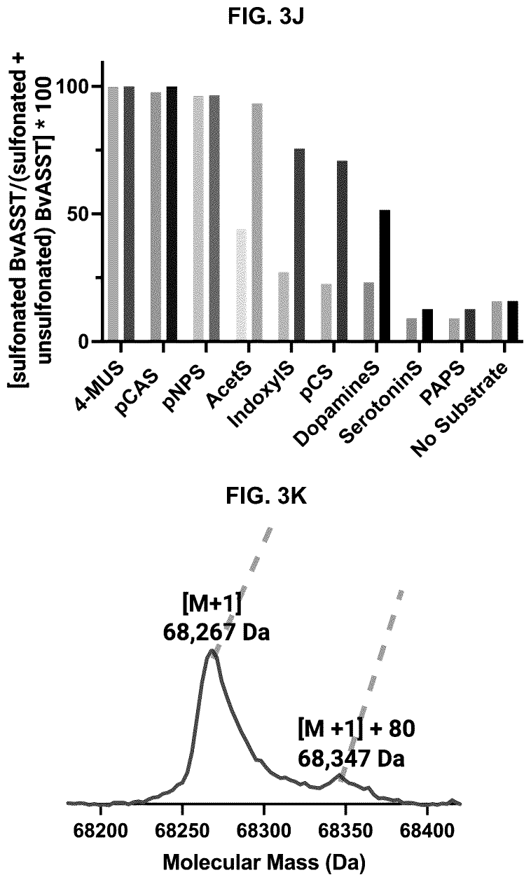

Interestingly, in the presence of most of the above-mentioned phenolic sulfates, we found a mass shift of +80 Da to produce a peak representing the sulfonated intermediate of BvASST with an intact mass of 68,347 Da (FIG. 3), demonstrating that sulfonation reactions were taking place. We did not detect a sulfonated form of BvASST when using serotoninS and PAPS as sulfo donors (FIG. 3), suggesting that neither molecule functions as a sulfo group donor for this enzyme. Although PAPS is a common sulfo donor for all hSULTs, it is not an aryl sulfate, which may explain its ineffectiveness with BvASST. SerotoninS, despite being an aryl sulfate, also failed to transfer a sulfo group possibly due to its amine characteristic. To better understand the structural and chemical requirements of sulfo donors for BvASST, additional testing with molecules structurally similar to serotoninS is needed. These results showed that exogenous and endogenous phenolic sulfates with biological relevance to humans are sulfo donors for BvASST. In agreement with other ASSTs, BvASST was also sulfonated in the presence of synthetic sulfo donors (FIG. 3).

Additionally, the screen to identify phenolic sulfo donors was carried out with two separate concentrations of donor molecules against BvASST, 100 μM (Table 3) and 1,000 μM (1 mM, Table 4) (FIG. 3).

4-MUS (FIG. 3A), pCAS (FIG. 3B), and pNPS (FIG. 3C) fully sulfonated BvASST at only 100 μM as apparent by the complete shift of the mass peak [M+1] (68,267 Da) to [M+1]+80 (68,347 Da; FIGS. 3A-C and K, spectra with lighter shades) with no further increase in [M+1]+80 (68,347 Da) peak which represents the sulfonated BvASST when donor concentrations were increased to 1 mM (FIG. 3A-C, spectra with darker shades). In contrast, BvASST was only partially sulfonated at 100 μM as apparent with the presence of two peaks ([M+1] which is unsulfonated form and [M+1]+80 which is sulfonated form) with acetS (FIG. 3D, lighter shade spectrum), indoxylS (FIG. 3E, lighter shade spectrum), pCS (FIG. 3F, lighter shade spectrum), and dopamineS (FIG. 3G, lighter shade spectrum). When incubated with 1 mM, there was an increase in the mass peak ([M+1]+80, 68,347 Da) representing sulfonated BvASST with concomitant decrease in the mass peak ([M+1], 68,267 Da) corresponding to unsulfonated BvASST in the presence of acetS (FIG. 3D, darker spectrum), indoxylS (FIG. 3E, darker spectrum), pCS (FIG. 3F, darker spectrum), and dopamineS (FIG. 3G, darker spectrum). We did not observe any mass shift of the [M+1] peak (unsulfonated form) where BvASST was incubated with either serotoninS (FIG. 3H) or PAPS (FIG. 3I), indicating that these molecules could not transfer sulfo group to BvASST and hence cannot be donors for BvASST. These differences in sulfonation process via various molecules are likely driven by the donor preferences of BvASST.

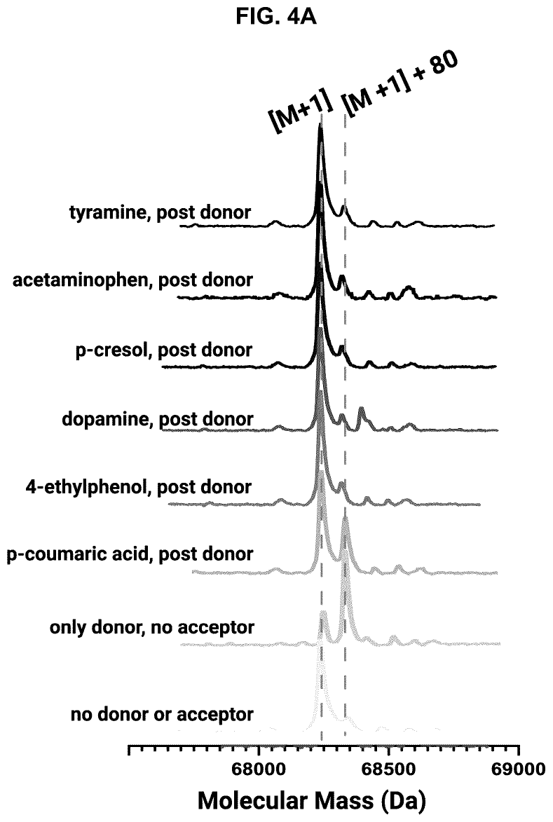

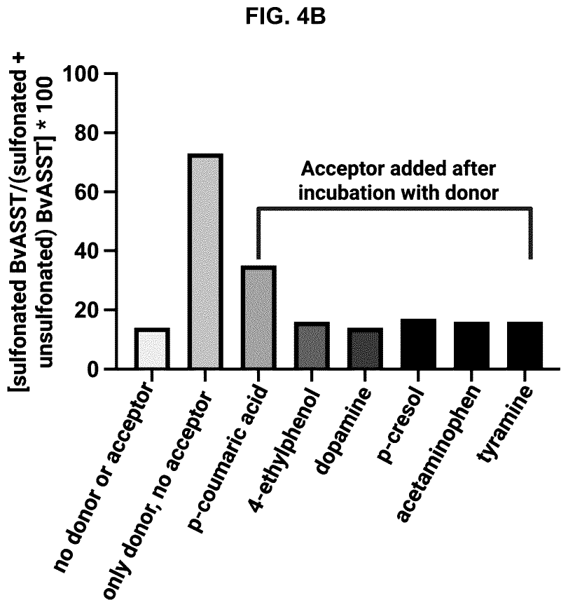

As per predicted mechanism, the sulfonated enzyme intermediate (sulfonated BvASST) transfers sulfo group to potential acceptors and returns to its original form (unsulfonated BvASST) (FIG. 2). To screen possible sulfo acceptors, BvASST was first converted to its sulfonated form by the addition of the donor, pNPS. BvASST incubated with pNPS (only donor, no acceptor) showed a large proportion of sulfonated ([M+1]+80) form when compared with BvASST incubated without any substrate (no donor, no acceptor; FIG. 4). Here, we used equimolar concentrations of pNPS and BvASST, aiming to achieve a single-turnover reaction to observe only the unsulfonation of the enzyme in the presence of potential acceptors. If pNPS was in excess, we hypothesized that the enzyme would predominantly remain in its sulfonated form and would interfere with our assay to screen acceptors. However, further analysis revealed that the pNPS concentration was slightly lower than that of BvASST, which likely explains incomplete sulfonation of the enzyme in contrast to full sulfonation observed in FIG. 3.

When sulfonated BvASST was incubated with various sulfo acceptors, we observed a decrease in sulfonated enzyme peak and a corresponding increase in the unsulfonated form (FIG. 4A), suggesting possible transfer of the sulfo group from the enzyme to an acceptor molecule. However, this assay does not directly detect the formation of sulfated acceptor products which must be confirmed through additional experimental validation. FIG. 4B provides a schematic representation of the qualitative trends observed in the intact mass spectrometry data from FIG. 4A. This assay was designed to screen potential sulfo acceptors for BvASST rather than to compare their relative efficiencies. For all tested acceptor molecules, a decrease in enzyme sulfonation was evident (FIG. 4A). We observed peak intensity shifts in the presence of tyramine, dopamine, pC, 4-EP, acetaminophen, and pCA, indicating that each of these molecules facilitated the conversion of sulfonated BvASST back to its primarily unsulfonated form, making it available for additional catalytic cycles. While we are not quantitatively comparing acceptor efficiencies, pCA exhibited the smallest decrease in sulfonation, suggesting possible reaction reversibility and equilibrium effects leading to the accumulation of both enzyme forms, though this requires further experimental validation. Collectively, intact mass-spectrometry data (FIG. 3-4) confirmed both half reactions of BvASST-catalyzed sulfo transfer and identified various phenolic compounds as sulfo donors and acceptors for this enzyme.

### BvASST is Sulfonated at a Histidine Residue

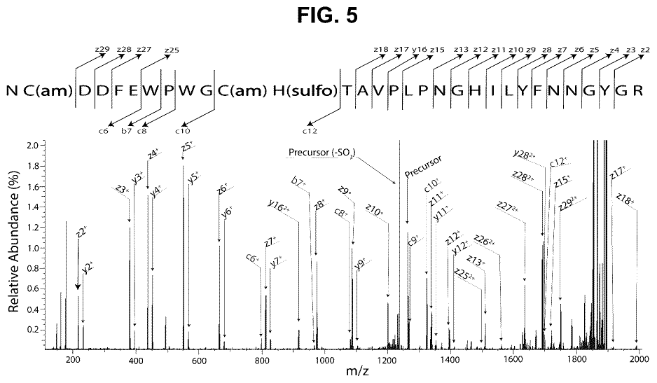

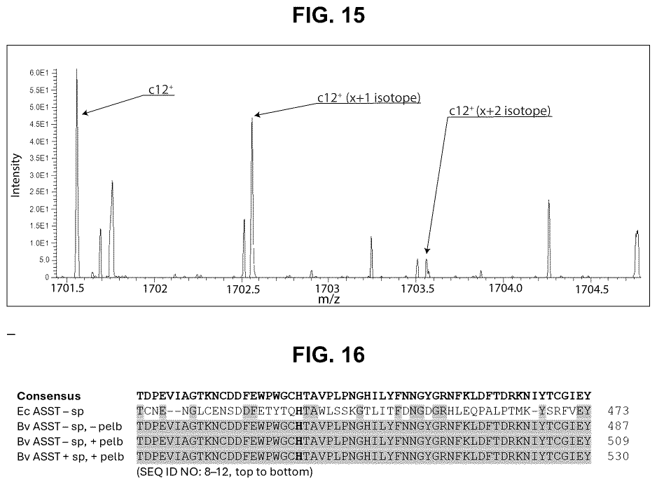

To identify the site of sulfonation, BvASST underwent trypsin digestion and the peptide that contained a sulfonated residue was identified by LC-MS/MS (FIG. 5). An amino acid residue sulfonated in the reaction catalyzed by BvASST was identified as a histidine residue, specifically H471 (FIG. 15). This residue is conserved within ASSTs (FIG. 16) and was identified as the site of sulfonation in an ASST from a uropathogenic strain of an E. coli.

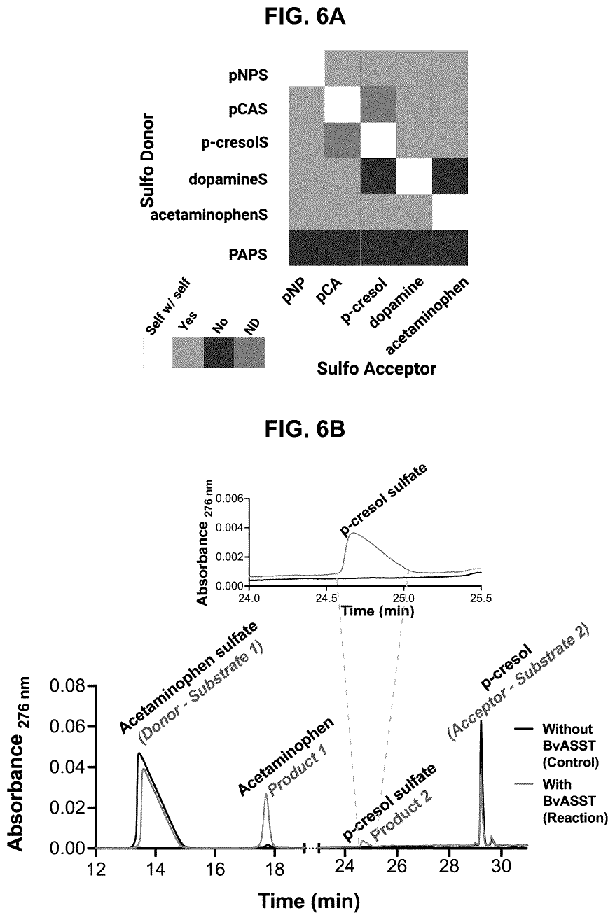

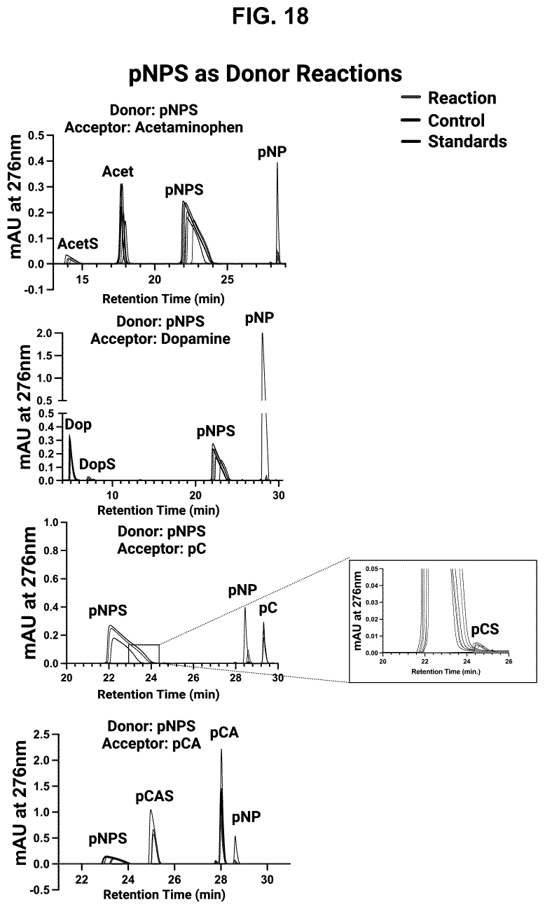

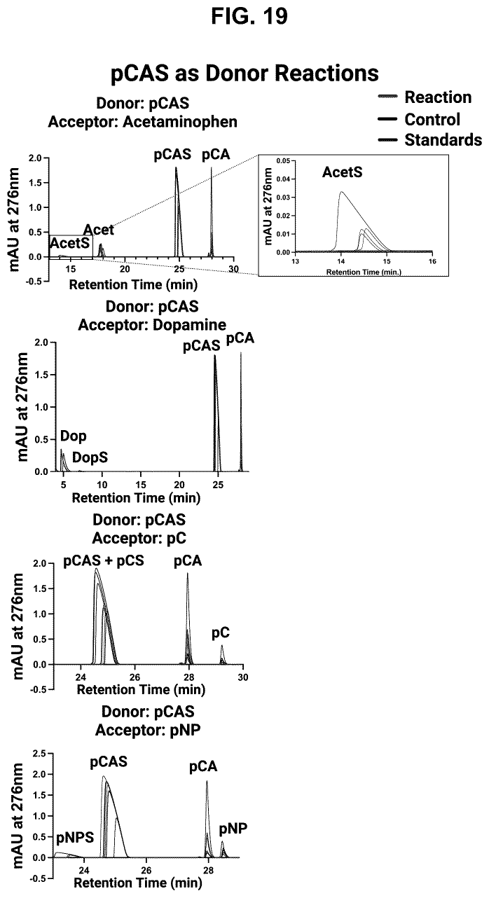

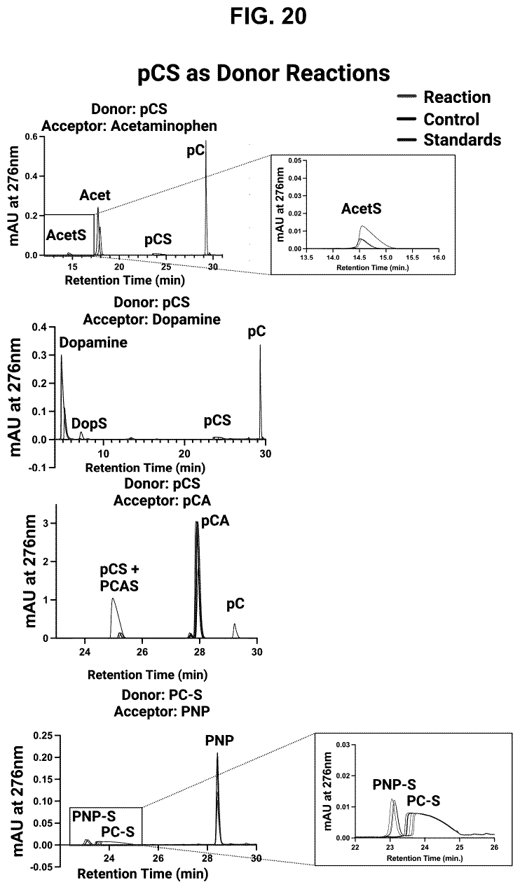

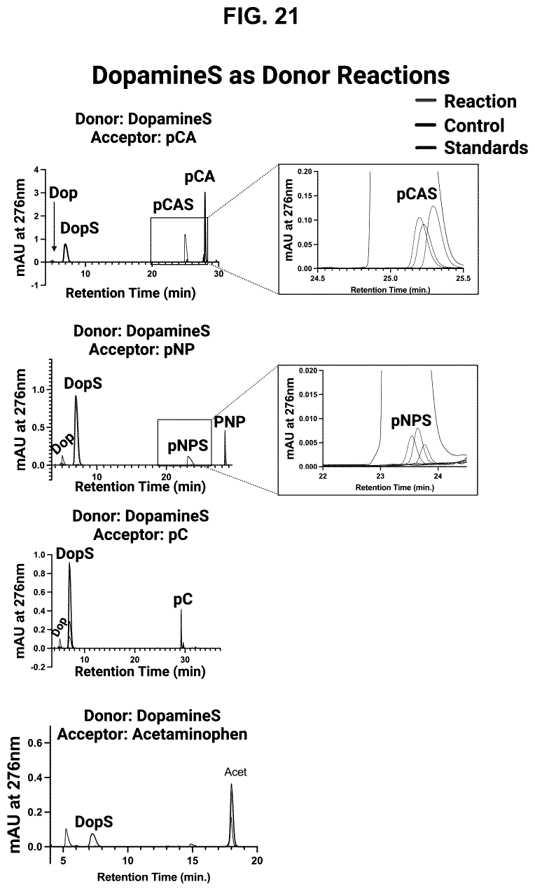

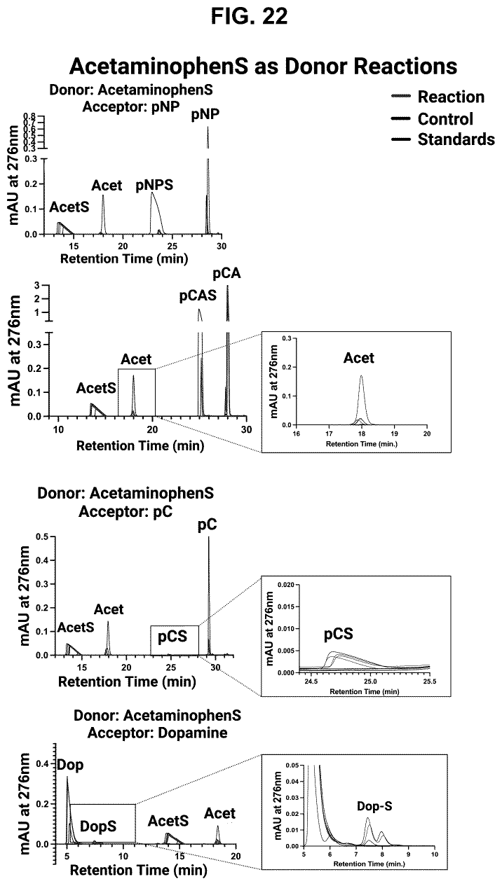

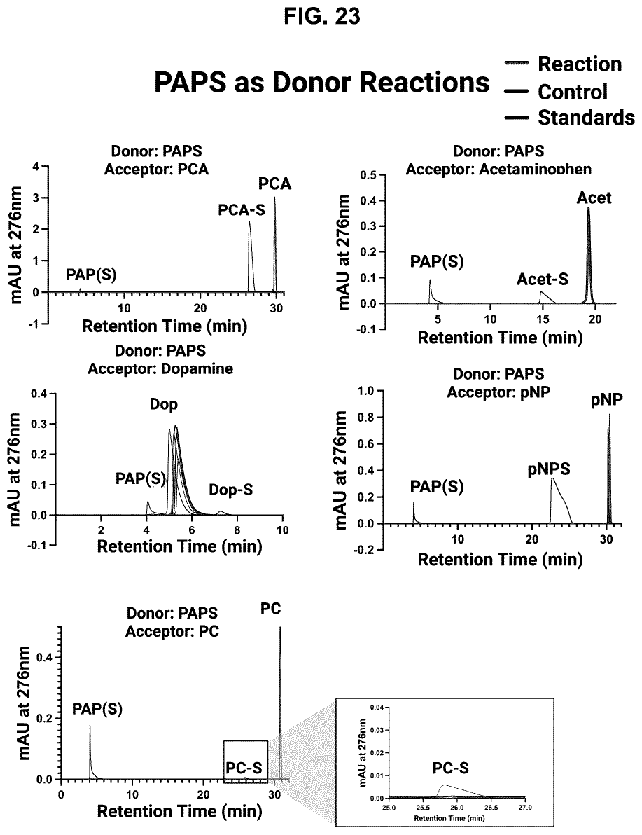

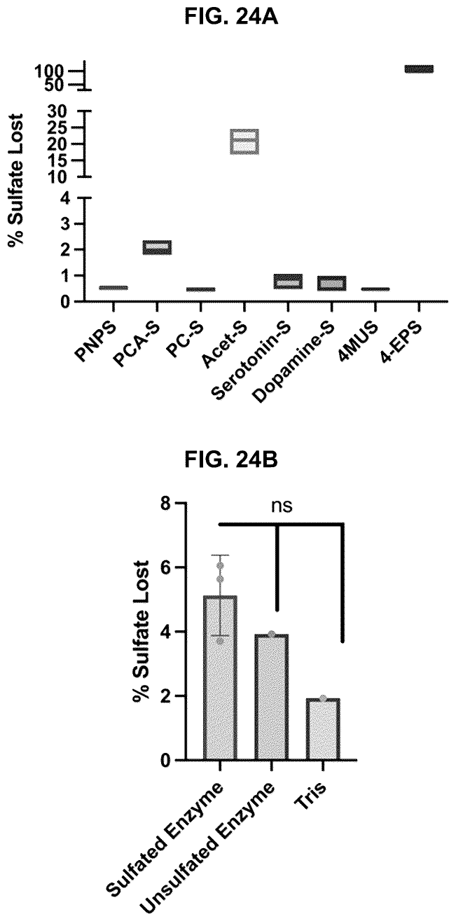

In vitro enzymatic assays with BvASST show sulfo transfer reactions between various donor-acceptor pairs of biologically relevant phenolic compounds. Following the identification of donor and acceptor substrates for BvASST, we tested various donor-acceptor pairs to assess their compatibility and determine if reaction products could be detected. To probe this, we provided BvASST with acetS, pCAS, pCS, dopamineS, PAPS, and pNPS as sulfo donor substrates (FIG. 6). We tested each of these sulfo donor substrates with several acceptor substrates that include dopamine, pC, pCA, pNP, and acetaminophen (FIG. 6). Interestingly, we detected the production of sulfated phenolic acceptor molecules and desulfated phenolic donor molecules in many reaction pairs (FIG. 6A). No products were detected when dopamineS was utilized as a donor with pC and acetaminophen as acceptors. Additionally, we did not observe any sulfated products where PAPS was the sulfo donor, confirming that BvASST is PAPS-independent sulfotransferase. Dopamine, a neurotransmitter, was a universal acceptor for all tested donors and these reactions produced dopamineS (FIG. 18-23). In contrast, acetS was found to be the universal donor (FIG. 22), where sulfonated forms of all phenolic acceptors that include pCA, pC, dopamine, and pNP were detected along with desulfonated acetaminophen (FIGS. 6A and 22). One such reaction has been depicted in FIG. 6B, where acetS was reacted with pC. In humans, one of the ways to metabolize acetaminophen is via sulfonation, and hence, acetS is a xenobiotic metabolite, whereas pC is only produced by gut microbes. Here, we showed that purified BvASST catalyzed a sulfo transfer reaction between acetS and pC to generate acetaminophen and pCS (FIG. 6B). This is an interesting chemistry that might have larger implications. For example, children with ASD have difficulty with the metabolism of the commonly used analgesic and antipyretic agent, acetaminophen. This has partly been attributed to the competition in sulfonation between various acceptors during sulfo transfer reactions catalyzed by hSULTs. It has been hypothesized that pC and acetaminophen compete for sulfonation and that excessive pCS formation interferes with the sulfonation pathway for acetaminophen, one of the principal routes for its metabolism and excretion. These observations highlight the need for a deeper understanding regarding the human gut microbiome's ability to alter the concentration of these compounds. Our results demonstrate that diverse phenolic molecules and phenolic sulfates which are known to be biologically relevant can be transformed by BvASST in vitro.

To better understand their interactions with other phenolic molecules, this study also included pNP and pNPS which are synthetic, nonphysiological acceptor and donor substrates, respectively. All donor molecules, except PAPS, were able to sulfonate pNP (FIG. 18-23). Similarly, all phenolic acceptor substrates were successfully sulfonated by the nonphysiological donor pNPS (FIG. 18). Due to their broad reactivity, pNP and pNPS can serve as reliable positive controls in BvASST-catalyzed reactions, helping to ensure the experimental setup is functioning optimally.

### Continuous Kinetic Assays Identify Potential Additional Phenolic Sulfo Group Acceptors for BvASST-Catalyzed Reactions

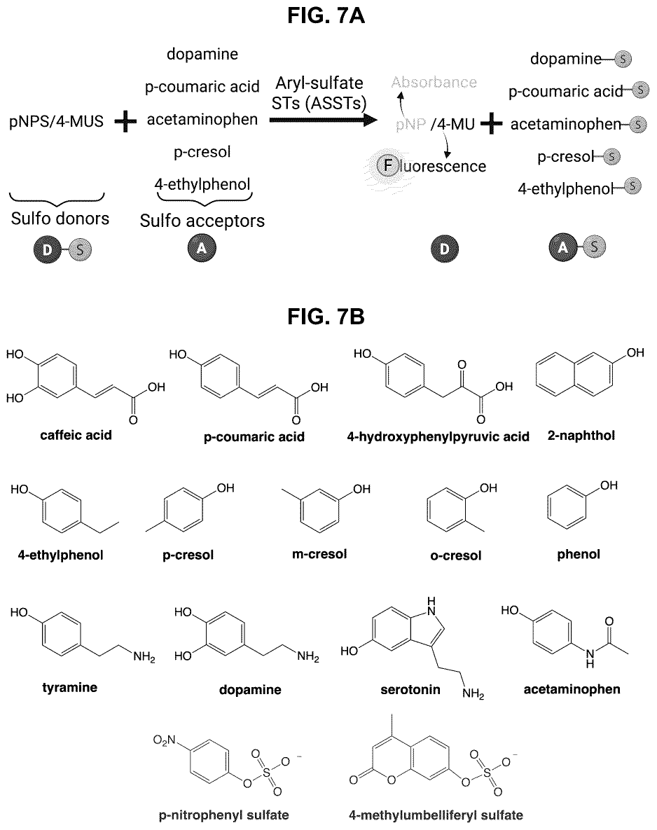

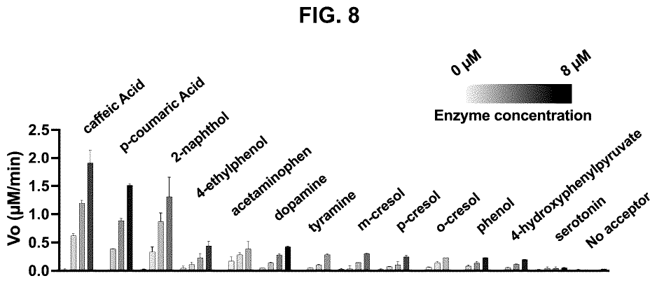

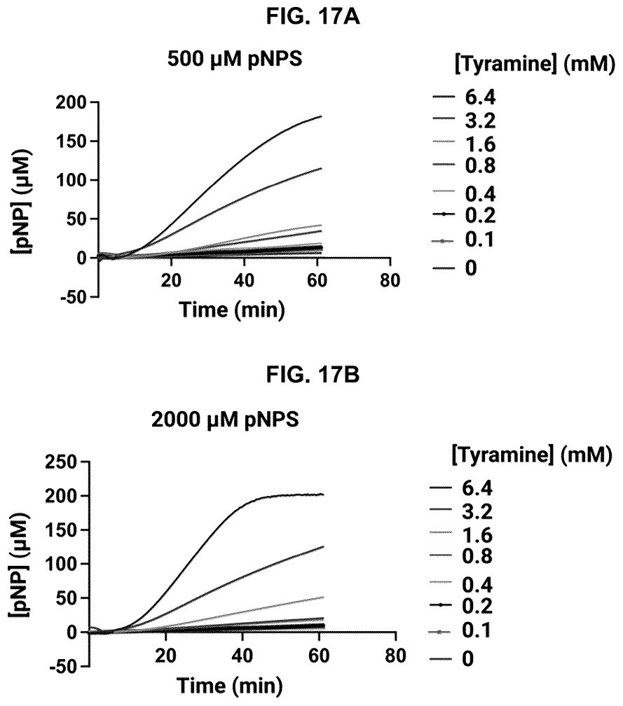

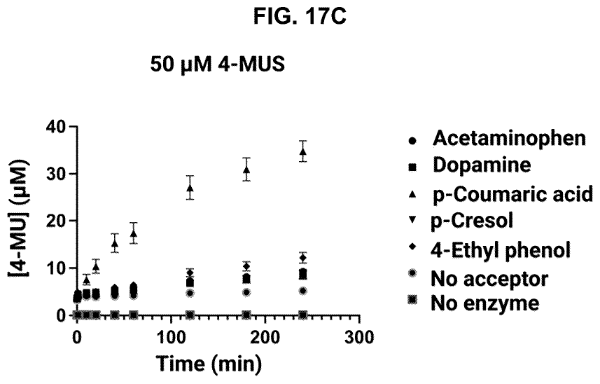

To test whether BvASST can utilize additional phenolic molecules as acceptors apart from above tested compounds (FIG. 4) in vitro, we expanded our library of phenolic acceptors and reacted them with BvASST. Phenolic molecules shown in FIG. 7B, including caffeic acid, serotonin, phenol, o-cresol, m-cresol, 2-naphthol, and 4-hydroxyphenylpyruvate (4-HPP) were tested as acceptors for the reaction catalyzed by BvASST. To test these multitude of phenolic acceptors, enzymatic activity assays shown in FIG. 8 were conducted with synthetic sulfo donors, pNPS (for colorimetric assays), and 4-MUS (for fluorescence-based assays; FIGS. 7A and 17) which provided continuous, stable, reliable, and high-throughput measurements for all molecules illustrated in FIG. 7B (Table 5).

TABLE 5
Initial velocities with varying enzyme concentrations (see FIG. 8)

| | Velocities (μM/min) with Carious | | | |
|---|---|---|---|---|
| Acceptor | Concentrations of BvASST | | | |
| Molecule | 0 μM | 2 μM | 4 μM | 8 μM |
| phenol | 0.01447 | 0.09496 | 0.1214 | 0.2309 |
| phenol | 0.01447 | 0.07170 | 0.1552 | 0.2334 |
| p-cresol | 0.01597 | 0.07660 | 0.1452 | 0.2687 |
| p-cresol | 0.03194 | 0.06964 | 0.1030 | 0.2272 |
| o-cresol | −0.02302 (0) | 0.05993 | 0.1271 | 0.2318 |
| o-cresol | −0.002381 (0) | 0.06774 | 0.1573 | 0.2330 |
| m-cresol | 0.03457 | 0.07092 | 0.1464 | 0.3093 |
| m-cresol | 0.02272 | −0.05507 (0) | 0.1458 | 0.2976 |
| caffeic acid | 0.02844 | 0.5949 | 1.232 | 2.072 |
| caffeic acid | 0.005853 | 0.6462 | 1.161 | 1.746 |
| 2-napthol | 0.02288 | 0.2787 | 0.7674 | 1.554 |
| 2-napthol | 0.02728 | 0.3966 | 0.9788 | 1.066 |
| 4-ethyl phenol | 0.07415 | 0.08526 | 0.1793 | 0.3809 |
| 4-ethyl phenol | 0.02212 | 0.1383 | 0.2822 | 0.4989 |
| no acceptor | 0.02319 | 0.01493 | 0.01681 | 0.03343 |
| no acceptor | −0.0008433 (0) | −0.0008533 (0) | 0 | 0 |
| acetaminophen | −3.512 × 10⁻⁵ (0) | 0.2241 | 0.3074 | 0.4810 |
| acetaminophen | 0.0004569 | 0.1186 | 0.2591 | 0.2966 |
| tyramine | 0.000333 | 0.05471 | 0.1101 | 0.2959 |
| tyramine | 0.0005196 | 0.05177 | 0.09501 | 0.2736 |
| dopamine | 0.04898 | 0.1319 | 0.2950 | 0.4203 |
| dopamine | 0.04850 | 0.1498 | 0.2680 | 0.4325 |
| p-coumaric acid | 0.002247 | 0.3838 | 0.9145 | 1.497 |
| p-coumaric acid | 0.001155 | 0.3889 | 0.8596 | 1.532 |
| serotonin | 0.02520 | 0.02722 | 0.02722 | 0.05187 |
| serotonin | 0.01948 | 0.06145 | 0.06145 | 0.05839 |
| hydroxy indole | −0.05851 (0) | 0.1258 | 0.1827 | 0.1611 |
| hydroxy indole | −0.01257 (0) | 0.1817 | 0.2579 | 0.1564 |
| 4-HPP | 0.0009441 | 0.05454 | 0.1187 | 0.1984 |
| 4-HPP | −0.001787 (0) | 0.05928 | 0.1100 | 0.2023 |

Initial velocities measured for BvASST catalyzed reactions between pNPS and various acceptors.
Measured through the production of pNP at 405 nm.

When pNPS was used as the sulfo donor, the reaction velocity for most molecules increased linearly with increasing BvASST concentration (FIG. 8). This confirmed that the observed product formation was due to enzymatic activity rather than spontaneous or noncatalytic processes and BvASST was able to use these molecules as sulfo group acceptors. For serotonin, little to no activity was observed in the presence of BvASST. Moreover, initial velocities changed significantly depending on the type of the phenolic molecule utilized as an acceptor.

The phenolic molecule with the highest velocity was the preferred acceptor for BvASST under our assay conditions. We observed that caffeic acid exhibited the highest reaction rates, followed by pCA. Both molecules are structurally similar, differing only by the presence of a hydroxyl group (FIG. 7B, structures 1 and 2).

### Ranking of Acceptor Compatibility Remained Similar Between the Two Synthetic Donors

As described previously, BvASST is capable of utilizing a broad range of acceptor substrates. To evaluate acceptor preference of BvASST, we conducted steady-state kinetic assays (FIG. 7A) in which the sulfo donor was held constant and the acceptor concentration was systematically varied, enabling us to determine apparent kinetic parameters for several sulfo acceptors, including dopamine, acetaminophen, pC (4-methylphenol), 4-EP, caffeic acid, and pCA. The apparent kinetic parameters varied widely for these acceptor molecules. The preference for acceptor was ranked by kcat/Km, which describes catalytic efficiency. The relative acceptor ranking for caffeic acid, pCA, and dopamine was consistent with either sulfo donor-pNPS (Table 6) or 4-MUS (Table 7).

TABLE 6
Steady-state Kinetic Parameters for BvASST with
Various Acceptors using pNPS as a Sulfo Group

| | K_{M} | k_{cat} | k_{cat}/K_{M} |
|---|---|---|---|
| Acceptor | (μM) | (min⁻¹) | (μM⁻¹ min⁻¹) |
| Caffeic acid | 22.2 ± 2.7 | 0.49 ± 0.01 | 0.0220 ± 0.00240 |
| n = 3 | | | |
| p-Coumaric acid | 38.4 ± 2.3 | 0.80 ± 0.06 | 0.0210 ± 0.00030 |
| n = 3 | | | |
| Dopamine | 2,008 ± 217 | 0.41 ± 0.01 | 0.0002 ± 0.00002 |
| n = 3 | | | |
| 4-Ethylphenol | ND—unable | ND—unable | |
| n = 3 | to saturate | to saturate | |
| p-Cresol | ND—unable | ND—unable | |
| n = 3 | to saturate | to saturate | |
| Acetaminophen | ND—unable | ND—unable | |
| n = 3 | to saturate | to saturate | |

Apparent kinetic parameters for different sulfo acceptors using pNPS as the sulfo donor. Measured via pNP production by monitoring absorbance at 405 nm (ε = 18,000 M⁻¹ cm⁻¹). Values are expressed as mean ± SD.

TABLE 7
Steady-State Kinetic Parameters for BvASST with Various
Acceptors using 4-MUS as a Sulfo Group Donor

| | K_{M} | k_{cat} | k_{cat}/K_{M} |
|---|---|---|---|
| Acceptor | (μM) | (min⁻¹) | (μM⁻¹ min⁻¹) |
| Caffeic acid | 55 ± 21 | 0.045 ± 0.006 | 0.00088 ± 0.000260 |
| n = 3 | | | |
| p-Coumaric acid | 131 ± 46 | 0.104 ± 0.007 | 0.00085 ± 0.000250 |
| n = 2 | | | |
| Dopamine | 2,292 ± 711 | 0.071 ± 0.016 | 0.00003 ± 0.000002 |
| n = 3 | | | |
| 4-Ethylphenol | 756 ± 77 | 0.087 ± 0.004 | 0.00012 ± 0.000006 |
| n = 2 | | | |
| p-Cresol | 2,712 ± 169 | 0.101 ± 0.006 | 0.00004 ± 0.000001 |
| n = 3 | | | |
| Acetaminophen | ND—unable | ND—unable | |
| n = 3 | to saturate | to saturate | |

Apparent kinetic parameters for different sulfo acceptors using 4-MUS as the sulfo donor. Measured via 4-MU production by monitoring fluorescence at 460 nm (excitation 360 nm). Concentrations were determined with the help of a standard curve. Values are expressed as mean ± SD.

For reactions where pNPS was utilized as a donor, acceptors were ranked as: caffeic acid≥pCA>dopamine. For reactions with 4-MUS as a donor, acceptors were ranked as: caffeic acid≥pCA>4-EP>pC≥dopamine. To determine whether acceptor preference is an intrinsic feature of the enzyme, it will be important to test these acceptors with the newly identified phenolic sulfo donors mentioned above whose modulation is known to influence human health. In the BvASST-catalyzed reactions between synthetic sulfo donors (both 4-MUS and pNPS) and acetaminophen, the rate of reaction increased linearly with rising acetaminophen concentrations, without saturation within the substrate solubility limits. Similar kinetic behavior with a linear trend appeared when pNPS was the donor and either 4-EP or pC served as acceptors. This absence of saturation could possibly indicate that all tested acceptor concentrations were below the apparent K_{M} or that the reactions may be partially reversible.

Conjugation reactions like sulfonation play a key role in the metabolism and excretion of phenolic compounds. Many biologically important metabolites fall into this category, including phenolic sulfates. Altered levels of phenolic compounds have been linked to various disorders. For instance, several studies have reported gut microbial dysbiosis and differential abundance of both sulfated and nonsulfated phenolic compounds including phenol, pC, pCS, and 4-ethylphenyl sulfate (4-EPS) in the fecal, urine, or serum samples of individuals with ASD. Moreover, sulfated derivatives of phenolic compounds like pCS and 4-EPS have been shown to exert adverse effects on the host with pCS contributing to chronic kidney disease and increased cardiovascular disease risk in mice and modulation of drug metabolism in humans, whereas 4-EPS exhibited anxiety-like behaviors in mice. Dopamine, a neurotransmitter produced in large amounts in the gastrointestinal tract, has also been implicated in the pathophysiology of Alzheimer's disease, Parkinson's disease, and obesity. Therefore, understanding the factors that regulate sulfonation of these phenolic compounds is crucial.

To investigate the potential role of the human gut microbiome in the sulfonation of small phenolic compounds, we performed a bioinformatics analysis to identify putative genes annotated to encode ASST enzymes which catalyze sulfo group transfer reactions involving phenolic substrates, within prevalent gut microbial species. To probe catalytic function and to identify substrates, we selected a representative ASST (BvASST) from the abundant gut microbe B. vulgatus, according to the criteria outlined earlier. Interestingly, many Bacteroides species contain a sulfotransferase gene encoding a SULT homolog, which is believed to function as a PAPS-dependent sulfotransferase. Unlike most Bacteroides species, B. vulgatus is unique in harboring two distinct sulfotransferase genes, one encoding a potential PAPS-dependent sulfotransferase (SULT homolog) as previously reported in B. thetaiotaomicron, and the other annotated as an ASST (BvASST). The reason for the coexistence of these two sulfotransferases in B. vulgatus and their respective contributions to microbial and host physiology remains unclear. Interestingly, we found that when expressed in E. coli, BvASST exhibits lipoprotein properties which is an unusual characteristic, as none of the previously studied ASSTs have been identified as lipoproteins.

With this study, we demonstrated that BvASST can utilize a wide range of structurally diverse phenolic compounds as sulfo group acceptors. These include phenolic acids (e.g., caffeic acid, pCA, 4-HPP), phenolic amines (e.g., dopamine, tyramine), phenolic amides (e.g., acetaminophen), simple phenolics (e.g., phenol, p/o/m-cresols, 4-EP), and a bicyclic phenol (2-naphthol). Interestingly, these molecules vary not only in their chemical classifications but also in their sources of origin. For example, we observed catalysis with dietary compounds like caffeic acid and pCA; microbially produced compounds such as pC, pCA, and 4-EP; synthetic xenobiotics like acetaminophen; host- or microbiota-derived molecules such as tyramine, dopamine, and phenol; host metabolic intermediates like 4-HPP; and naturally occurring compounds like 2-naphthol, a known precursor to several human-associated xenobiotics. These findings highlight the catalytic versatility of BvASST, which is capable of recognizing and modifying a broad spectrum of phenolic substrates in vitro with diverse chemical structures and biological origins.

Unlike PAPS-dependent sulfotransferases such as cytosolic hSULTs and microbial SULTs which utilize PAPS as a sulfo group donor, the identities of sulfo donors for ASSTs have remained largely unknown. Using intact mass spectrometry and HPLC, we identified several sulfo group donors for BvASST in vitro. These included biologically relevant sulfated metabolites such as acetS, dopamineS, pCAS, and pCS, compounds whose levels are known to influence host health. If such sulfo transfer reactions occur in the gut environment, it raises the possibility that gut microbial ASSTs may contribute to or modulate host metabolite pools. For instance, the sulfonation of acetaminophen is generally attributed to human sulfotransferases for its detoxification and excretion. However, our results show that BvASST can catalyze a similar reaction in vitro, suggesting that gut microbes may either complement or interfere with this host detoxification pathway. If this activity occurs in vivo, it could potentially alter the pharmacokinetics of acetaminophen by affecting its clearance rate and influencing optimal dosing, as a result of gut microbial enzyme activity. In such cases, microbial contributions to acetaminophen metabolism could have important implications for drug efficacy and toxicity. However, further in vivo studies are needed to experimentally validate this hypothesis.

Consistent with other characterized ASSTs, BvASST was capable of utilizing synthetic sulfo donors that are not naturally found in biological systems or environmental contexts. While these donors are unlikely to be encountered by the enzyme in vivo, they serve as useful tools for assessing enzymatic activity due to the colorimetric or fluorometric properties of their nonsulfated counterparts. The ability of BvASST to act on such non-natural substrates likely reflects coincidental rather than evolutionarily selected activity. This observation further suggests a degree of active site plasticity, enabling BvASST to accommodate and transform phenolic molecules that are potentially non-native to the organism.

In summary, we have identified a microbial sulfotransferase from a human commensal gut microbe that functions as a versatile catalyst. BvASST accepts a broad range of phenolic substrates and utilizes previously unrecognized sulfo donors to modulate the levels of phenolic metabolites in vitro that have known relevance to the gut-brain axis and overall human health. Future studies should aim to elucidate the physiological significance of these BvASST mediated transformations in vivo and could further explore whether BvASST and its homologs are actively expressed in the human gut microbiome by analyzing publicly available metatranscriptomic datasets to strengthen our understanding of their in vivo relevance.

SEQUENCES

| | | SEQ ID |
|---|---|---|
| Name | Sequence (N→C or 5′→3′) | NO |
| Bv ASST NT | ATGAAAAGGAAAGGATATTATTTATGCTCTTTGTTGGTTATGATATTTGTATTTT | 1 |
| | TGTCATGTCGTGATGAAGAACAGGATTGTACCCAAGCTATGCAGGATATACAAAA | |
| | CCAGTTGAAGGAAAATGATTTATTGAAATCTGTTTCTCAAGAACGTGAACAATGT | |
| | GTTTTGTTATTCGAGTCCAATAAGATAATTTTTCCGCAAGAGTTAATCCAAAGTG | |
| | TAGATGTGGATGCGGAAAACTGGAAAACGACTTTGACATTTGCCAATGGAAGCAC | |
| | TTATGTTATACCTACCTTAGGTACTTCGATTGACAACTTGATATTGAGTTCTACA | |
| | GTCAATCCTTCCGGTTGCAATCCATTATCGGCCAGTGTAGTTGTAAAATTACCTG | |
| | TATTGGGACGCATTAAACTGATTGTACACAGCAAACCGGGAAAGCATACGCCTGA | |
| | CGTGGAATATACATTTAAAGATGTAGGATTAAAACAGAATATTCCGGTATTGGGA | |
| | CTTTATCCCAATTATAATAACCAGATCACTTTAATTTATACGGATTTGCAAGGAA | |
| | ATGAACGTGCTCGGTCCAATCTAAAGCTTCAGACGAAAACATTGGAAAGCAGAAG | |
| | GCTGCCTAAGGAAATCAGAGTGGTAAAGGCTCAATATGACCGAATGGAGCCAGGC | |
| | ATGAATTTGGTAAACTCCCCCGGACAGGATGAAACAGACACTTCTATCCCTTATA | |
| | TGATAGATGCAGACGGAGAAATCCGTTGGATTCTTGATTGGGAGAAGTCGGATGA | |
| | ACATCGATATATCGGAATCGGGTGCGGATTGATCCGGATGCAGAACGGACATTAT | |
| | ATGACCGGTGACGGAAACCATCATCGGATGGTGGAGGTGGATATGATGGGAAATA | |
| | CAATTCATAATTGGGATATGCTGGAAAGGGGATACACTATGCATCATGCCATATC | |
| | TCAAGATAAGCAAGGCAATATATTAGCGACTGTGAGCAAGACTTCCGCTAAAATC | |
| | GCCAATGGGAAAGATGTACGGATAAATGATTTTATTATTATGATGGATCCCGAAA | |
| | AAGGGGAGATATTGCAAGAATGGGATTTGGTGCACATGTTGGATTCGGCTCGCTA | |
| | TGGAATGACCGATTATGATCTAACCTCAGACCCTTTTGCACAAAGTGCCAGTAAT | |
| | TGGGCGCATAATAATGGTATAGTAGAGTGGGGAGATGATTATCTGGCTACAGCTC | |
| | GTTATCAAGGAATCTTTAAGTTTAATAAAGCTGGAGGTATTGAGTGGATTATATC | |
| | GCCTCATGGGTATTGGAGAGATAAATACTTGAAATTATTATTGAATCCTCTTCAT | |
| | GCCGACGGGACTCCTATTACTGATCCTGAGGTGATAGCCGGAACGAAGAATTGTG | |
| | ATGATTTTGAATGGCCTTGGGGATGTCATACAGCTGTCCCATTACCTAATGGACA | |
| | CATTCTTTATTTCAATAATGGTTATGGGCGTAACTTCAAACTGGATTTTACAGAC | |
| | AGGAAAAATATTTATACTTGCGGCATAGAATATGAAGTGGATGAAGTGAACCGTA | |
| | CGGTTCGCCAAGTTTGGCAGTATGGAAAAGAGCGTGGTGCCGCGTATTTTACTCC | |
| | GGCCCGTTCCGGGGTGCAATATTTGGAACAAACGGGTAACAGATTAATTTGTCCG | |
| | GGAATGAGCAATGTGCTTAGCAATGGCAATCGTGGTGCACGCGTGACAGAAGTAG | |
| | ATCCTGAAACACAGGATGTTGTGTTTGAGCTGGAATTGGAAGATGCGATTTACCA | |
| | GCGGGTTTACCGTATGTCTCTTTATCCTGAGAACCAATAA | |
| Bv ASST AA | MKRKGYYLCSLLVMIFVFLSCRDEEQDCTQAMQDIQNQLKENDLLKSVSQEREQC | 2 |
| | VLLFESNKIIFPQELIQSVDVDAENWKTTLTFANGSTYVIPTLGTSIDNLILSST | |
| | VNPSGCNPLSASVVVKLPVLGRIKLIVHSKPGKHTPDVEYTFKDVGLKQNIPVLG | |
| | LYPNYNNQITLIYTDLQGNERARSNLKLQTKTLESRRLPKEIRVVKAQYDRMEPG | |
| | MNLVNSPGQDETDTSIPYMIDADGEIRWILDWEKSDEHRYIGIGCGLIRMQNGHY | |
| | MTGDGNHHRMVEVDMMGNTIHNWDMLERGYTMHHAISQDKQGNILATVSKTSAKI | |
| | ANGKDVRINDFIIMMDPEKGEILQEWDLVHMLDSARYGMTDYDLTSDPFAQSASN | |
| | WAHNNGIVEWGDDYLATARYQGIFKFNKAGGIEWIISPHGYWRDKYLKLLLNPLH | |
| | ADGTPITDPEVIAGTKNCDDFEWPWGCHTAVPLPNGHILYENNGYGRNFKLDFTD | |
| | RKNIYTCGIEYEVDEVNRTVRQVWQYGKERGAAYFTPARSGVQYLEQTGNRLICP | |
| | GMSNVLSNGNRGARVTEVDPETQDVVFELELEDAIYQRVYRMSLYPENQ | |
| Bv ASST | ATGGCCATGAAACGCAAAGGCTATTATCTGTGCAGCCTGCTGGTGATGATTTTTG | 3 |
| Codon | TGTTTCTGAGCTGCCGCGATGAAGAACAAGATTGCACCCAAGCGATGCAAGATAT | |
| Optimized | TCAGAATCAGCTGAAAGAAAACGATCTGCTGAAAAGCGTGAGCCAAGAACGCGAA | |
| NT | CAGTGCGTGCTGCTGTTTGAAAGCAACAAAATTATTTTTCCGCAAGAACTGATTC | |
| | AGAGCGTGGATGTGGATGCGGAAAACTGGAAAACCACCCTGACCTTTGCTAACGG | |
| | CAGCACCTATGTGATTCCGACCCTGGGCACGAGCATTGATAACCTGATTCTGAGC | |
| | AGCACCGTGAACCCGAGCGGCTGCAACCCGCTGAGCGCGAGCGTGGTGGTGAAAC | |
| | TGCCCGTGCTGGGCCGCATTAAACTGATTGTGCATAGCAAACCGGGCAAACATAC | |
| | CCCGGATGTGGAATATACCTTTAAAGATGTGGGCCTGAAACAGAACATTCCGGTG | |
| | CTGGGCCTGTATCCGAACTATAACAATCAGATTACCCTGATTTATACCGATCTGC | |
| | AAGGCAACGAACGCGCGCGCAGCAACCTGAAACTGCAGACCAAAACCCTGGAAAG | |
| | CCGCCGCCTGCCGAAAGAAATTCGCGTGGTGAAAGCGCAGTATGATCGCATGGAA | |
| | CCGGGCATGAACCTGGTGAACAGCCCGGGCCAAGATGAAACCGATACGAGCATTC | |
| | CGTATATGATTGATGCGGATGGCGAAATTCGCTGGATTCTGGATTGGGAAAAAAG | |
| | CGATGAACATCGCTATATTGGCATTGGCTGCGGCCTGATTCGCATGCAGAACGGC | |
| | CATTATATGACCGGCGATGGCAACCATCATCGCATGGTGGAAGTGGATATGATGG | |
| | GCAACACCATTCATAACTGGGATATGCTGGAACGCGGCTATACCATGCATCATGC | |
| | GATTAGCCAAGATAAACAAGGCAACATTCTGGCGACCGTGAGCAAAACGAGCGCG | |
| | AAAATTGCGAACGGCAAAGATGTGCGCATTAACGATTTTATTATTATGATGGATC | |
| | CGGAGAAAGGAGAAATTCTGCAAGAATGGGATCTGGTGCATATGCTGGATAGCGC | |
| | GCGCTATGGCATGACCGATTATGATCTGACGAGCGATCCGTTTGCGCAGAGCGCG | |
| | AGCAACTGGGCGCATAACAACGGCATTGTGGAATGGGGCGATGATTATCTGGCGA | |
| | CCGCGCGCTATCAAGGCATTTTTAAATTTAACAAAGCGGGAGGCATTGAATGGAT | |
| | TATTAGCCCGCATGGCTATTGGCGCGATAAATATCTGAAACTGCTGCTGAACCCG | |
| | CTGCATGCGGATGGCACCCCGATTACCGATCCGGAAGTGATTGCGGGCACCAAAA | |
| | ACTGCGATGATTTTGAATGGCCGTGGGGCTGCCATACCGCGGTGCCGCTGCCGAA | |
| | CGGCCATATTCTGTATTTTAACAACGGCTATGGCCGCAACTTTAAACTGGATTTT | |
| | ACCGATCGCAAAAACATTTATACCTGCGGAATAGAATATGAAGTGGATGAAGTGA | |
| | ACCGCACCGTGCGCCAAGTGTGGCAGTATGGCAAAGAACGCGGCGCGGCGTATTT | |
| | TACCCCGGCGCGCAGCGGCGTGCAGTATCTGGAACAGACCGGCAACCGCCTGATT | |
| | TGCCCGGGCATGAGCAACGTGCTGAGCAACGGCAACCGCGGCGCGCGCGTGACCG | |
| | AAGTTGATCCGGAAACCCAAGATGTGGTGTTTGAACTGGAACTGGAAGATGCGAT | |
| | TTATCAGCGCGTGTATCGCATGAGCCTGTATCCGGAAAATCAGTAAGAGCTC | |
| Bv ASST | MAMKRKGYYLCSLLVMIFVFLSCRDEEQDCTQAMQDIQNQLKENDLLKSVSQERE | 4 |
| Codon | QCVLLFESNKIIFPQELIQSVDVDAENWKTTLTFANGSTYVIPTLGTSIDNLILS | |
| Optimized | STVNPSGCNPLSASVVVKLPVLGRIKLIVHSKPGKHTPDVEYTFKDVGLKQNIPV | |
| AA | LGLYPNYNNQITLIYTDLQGNERARSNLKLQTKTLESRRLPKEIRVVKAQYDRME | |
| | PGMNLVNSPGQDETDTSIPYMIDADGEIRWILDWEKSDEHRYIGIGCGLIRMQNG | |
| | HYMTGDGNHHRMVEVDMMGNTIHNWDMLERGYTMHHAISQDKQGNILATVSKTSA | |
| | KIANGKDVRINDFIIMMDPEKGEILQEWDLVHMLDSARYGMTDYDLTSDPFAQSA | |
| | SNWAHNNGIVEWGDDYLATARYQGIFKFNKAGGIEWIISPHGYWRDKYLKLLLNP | |
| | LHADGTPITDPEVIAGTKNCDDFEWPWGCHTAVPLPNGHILYFNNGYGRNFKLDF | |
| | TDRKNIYTCGIEYEVDEVNRTVRQVWQYGKERGAAYFTPARSGVQYLEQTGNRLI | |
| | CPGMSNVLSNGNRGARVTEVDPETQDVVFELELEDAIYQRVYRMSLYPENQ | |
| Bv ASST + | MKYLLPTAAAGLLLLLAAQPAMAMAMKRKGYYLCSLLVMIFVLSCRDEEQDCTQA | 5 |
| sp + pelb | MQDIQNQLKENDLLKSVSQEREQCVLLFESNKIFPQELIQSVDVDAENWKTTLTF | |
| | ANGSTYVIPTLGTSIDNILISSTVNPSGCNPLSASVVKLPVLGIRKLIVHSKPGK | |
| | HTPDVEYTFKDVGLKQNIPVIGLYPNYNNQITLIYTDLQGNERARSNLKLQTKTL | |
| | ESRRLDPKEIRVVKAQYDRMEPGMNLVNSPGQDETDTSIPYMIDADGEIRWLDWK | |
| | SDEHRYIGCGCLIRMQNGHYMTGDGDNHRMVEVDMMGNTIHNWDMLERGTMHHAI | |
| | SQDKQGNILATVSKTSAKIANGKDVRINDDFIIMMDPEKGEILQEWDLVHMLDSA | |
| | RYGMTDYDLTSDPFAQSASNWAHNNGIVEWGDDYLATARYQGIFKNKAGGIEWII | |
| | SPHGYWRDKYLKLLLNPLHADGTPITDPEVIAGTKNCDDDFEWPWGCHTAVPLPN | |
| | GHILYFNNGYGRNFKLDFTDRKNIYTCGIEYEVDEVNRTVRQVWQYGKERGAAYF | |
| | TPARSQVYLETGNRLICPGMSNVLSNGNRGARVTEVDPETQDVVFELELEDAIYQ | |
| | RVYRMSLYPENQ | |
| Bv ASST − | MKYLLPTAAAGLLLLLAAQPAMAMARDEEQDCTQAMQDIQNQLKENDLLKSVSQE | 6 |
| sp + pelb | REQCVLLFESNKIIFPQELIQSVDVDAENWKTTLTFANGSTYVIPTLGTSIDNLI | |
| | LSSTVNPSGCNPLSASVVKLPVLGIRKILIVHSKPGKHTPDVEYTFKDVGLKQNI | |
| | PVLGLYPNYNNQITLIYTDLQGNERARSNLKLQTKTLESRRLDPKEIRVKAQYDR | |
| | MEPGMNLVNSPGQDETDTSIPYMIDADGEIRWLDWKSDEHRYIGCGCLIRMQNGH | |
| | YMTGDGDNHRMVEVDMMGNTIHNWDMLERGTMHHAISQDKQGNILATVSKTSAKI | |
| | ANGKDVRINDDFIIMMDPEKGEILQEWDLVHMLDSARYGMTDYDLTSDPFAQSAS | |
| | NWAHNNGIVEWGDDYLATARYQGIFKNKAGGIEWIISPHGYWRDKYLKLLLNPLH | |
| | ADGTPITDPEVIAGTKNCDDDFEWPWGCHTAVPLPNGHILYFNNGYGRNFKLDFT | |
| | DRKNIYTCGIEYEVDEVNRTVRQVWQYGKERGAAYFTPARSGVYLETGNRLICPG | |
| | MSNVLSNGNRGARVTEVDPETQDVVFELELEDAIYQRVYRMSLYPENQ | |
| Bv ASST − | MARDEEQDCTQAMQDIQNQLKENDLLKSVSQEREQCVLLFESNKIFPQELIQSVD | 7 |
| sp − pelb | VDAENWKTTLTFANGSTYVIPTLGTSIDNILISSTVNPSGCNPLSASVVKLPVLG | |
| | IRKILIVHSKPGKHTPDVEYTFKDVGLKQNIPVIGLYPNYNNQITLIYTDLQGNE | |
| | RARSNLKLQTKTLESRRLPKEIRVVKAQYDRMEPGMNLVNSPGQDETDTSIPYMI | |
| | DADGEIRWLDWKSDEHRYIGICGCLIRMQNGHYMTGDGDNHRMVEVDMMGNTIHN | |
| | WDMLERGTMHHAISQDKQGNILATVSKTSAKIANGKDVRINDDFIIMMDPEKGEI | |
| | LQEWDLVHMLDSARYGMTDYDLTSDPFAQSASNWAHNNGIVEWGDDYLATARYQG | |
| | IFKNKAGGIEWIISPHGYWRDKYLKLLLNPLHADGTPITDPEVIAGTKNCDDDFE | |
| | WPWGCHTAVPLPNGHILYFNNGYGRNFKLDFTDRKNIYTCGIEYEVDEVNRTVRQ | |
| | VWQYGKERGAAYFTPARSQVYLEQTGNRLICPGMSNVLSNGNRGARVTEVDPETQ | |
| | DVVFELELEDAIYQRVYRMSLYPENQ | |
| Consensus | TDPEVIAGTKNCDDFEWPWGCHTAVPLPNGHILYENNGYGRNFKLDFTDRKNIYT | 8 |
| | CGIEY | |
| Ec ASST − | TCNENGLCENSDDFETYTQHTAWLSSKGTLITFDNGDGRHLEQPALPTMKYSRFV | 9 |
| SP | EY | |
| Bv ASST − | TDPEVIAGTKNCDDFEWPWGCHTAVPLPNGHILYFNNGYGRNFKLDFTDRKNIYT | 10 |
| sp, − pelb | CGIEY | |
| Bv ASST − | TDPEVIAGTKNCDDFEWPWGCHTAVPLPNGHILYFNNGYGRNFKLDFTDRKNIYT | 11 |
| sp, + pelb | CGIEY | |
| Ec ASST − | TDPEVIAGTKNCDDFEWPWGCHTAVPLPNGHILYFNNGYGRNFKLDFTDRKNIYT | 12 |
| SP | CGIEY | |
| BvASST Fwd | CCATGGCCCGCGATGAAGAACAAGATT | 13 |
| Primer | | |
| BvASST Rev | GAGCTCTTACTGATTTTCCGGATA | 14 |
| Primer | | |

What is claimed:

1. A method for identifying sulfo donor substrates for an aryl-sulfate sulfotransferase (ASST), the method comprising:
providing an ASST having an unsulfonated form;
incubating the ASST with a candidate sulfo donor compound; and
analyzing the ASST by intact protein mass spectrometry to detect conversion of the unsulfonated form to a sulfonated form, thereby identifying the candidate sulfo donor compound as a sulfo donor substrate for the ASST.

2. The method of claim 1, wherein the aryl-sulfate sulfotransferase is a 3′-phosphate-5′-phosphosulfate (PAPS) independent sulfotransferase.

3. The method of claim 1, wherein the unsulfonated form has a first molecular mass;
the sulfonated form has a second molecular mass that is approximately 80 Da greater than the first molecular mass; and
detecting the second molecular mass during the analyzing step identifies the candidate sulfo donor compound as a sulfo donor substrate for the ASST.

4. The method of claim 1, wherein the candidate sulfo donor compound comprises a phenolic sulfate or a non-phenolic sulfate.

5. The method of claim 1, wherein the candidate sulfo donor compound comprises acetaminophen sulfate, dopamine sulfate, p-coumaric acid sulfate, p-cresol sulfate, indoxyl sulfate, 4-methylumbelliferyl sulfate, or p-nitrophenyl sulfate.

6. The method of claim 1, further comprising isolating the ASST prior to the analyzing step.

7. The method of claim 4, wherein isolating the ASST comprises heterologous expression in E. coli, purification via column chromatography, and spin filtering.

8. The method of claim 1, wherein the ASST is a Bacteroides vulgatus ASST.

9. The method of claim 1, wherein the ASST has at least 90-99% identity to a polypeptide sequence selected from SEQ ID NO: 5-7.

10. The method of claim 1, wherein the ASST has a polypeptide sequence selected from SEQ ID NO: 5-7.

11. A method for identifying sulfo acceptor substrates for an aryl-sulfate sulfotransferase (ASST), the method comprising:
providing a sulfonated form of an ASST;
incubating the sulfonated form of the ASST with a candidate sulfo acceptor compound; and
analyzing the ASST by intact protein mass spectrometry to detect conversion of the sulfonated form to an unsulfonated form, thereby identifying the candidate sulfo acceptor compound as a sulfo acceptor substrate for the ASST.

12. The method of claim 11, wherein the aryl-sulfate sulfotransferase is a 3′-phosphate-5′-phosphosulfate (PAPS) independent sulfotransferase.

13. The method of claim 11, wherein the sulfonated form has a first molecular mass;
the unsulfonated form has a second molecular mass that is approximately 80 Da less than the first molecular mass; and
detecting the second molecular mass during the analyzing step identifies the candidate sulfo acceptor compound as a sulfo acceptor substrate for the ASST.

14. The method of claim 11, wherein the candidate sulfo donor compound comprises a phenolic sulfate or a non-phenolic sulfate.

15. The method of claim 11, further comprising isolating the ASST prior to the analyzing step.

16. The method of claim 11, wherein isolating the ASST comprises heterologous expression in E. coli, purification via column chromatography, and spin filtering.

17. The method of claim 11, further comprising:
incubating the ASST in the unsulfonated form with a sulfo group donor, thereby converting it to the sulfonated form; and
isolating the sulfonated form of the ASST;
wherein incubating the ASST in the unsulfonated form with the sulfo group donor and isolating the sulfonated form are performed prior to providing the sulfonated form of the ASST.

18. The method of claim 17, wherein the sulfo group donor is selected from the group consisting of acetaminophen sulfate, dopamine sulfate, p-coumaric acid sulfate, p-cresol sulfate, indoxyl sulfate, 4-methylumbelliferyl sulfate, and p-nitrophenyl sulfate.

19. The method of claim 11, wherein the ASST is a Bacteroides vulgatus ASST.

20. The method of claim 11, wherein the ASST has at least 90-99% identity to a polypeptide sequence selected from SEQ ID NO: 5-7.

21. The method of claim 11, wherein the ASST has a polypeptide sequence selected from SEQ ID NO: 5-7.

22. A modified Bacteroides vulgatus ASST having at least 90-99% identity to a polypeptide sequence selected from SEQ ID NO: 5-7.

23. The modified Bacteroides vulgatus ASST of claim 22, wherein the ASST has a polypeptide sequence selected from SEQ ID NO: 5-7.

24. The modified Bacteroides vulgatus ASST of claim 22, further comprising a PelB leader sequence or a signal peptide.


SEQUENCE LISTING (14 sequences)

SEQ ID NO: 1 - DNA, 1800 bp, unidentified
  source 1..1800: mol_type = other DNA; note = Organism - Bacteroides vulgatus ATCC 8482
atgaaaaggaaaggatattatttatgctctttgttggttatgatatttgtatttttgtcatgtcgtgatgaagaacaggattgtacccaagctatgcaggatatacaaaaccagttgaaggaaaatgatttattgaaatctgtttctcaagaacgtgaacaatgtgttttgttattcgagtccaataagataatttttccgcaagagttaatccaaagtgtagatgtggatgcggaaaactggaaaacgactttgacatttgccaatggaagcacttatgttatacctaccttaggtacttcgattgacaacttgatattgagttctacagtcaatccttccggttgcaatccattatcggccagtgtagttgtaaaattacctgtattgggacgcattaaactgattgtacacagcaaaccgggaaagcatacgcctgacgtggaatatacatttaaagatgtaggattaaaacagaatattccggtattgggactttatcccaattataataaccagatcactttaatttatacggatttgcaaggaaatgaacgtgctcggtccaatctaaagcttcagacgaaaacattggaaagcagaaggctgcctaaggaaatcagagtggtaaaggctcaatatgaccgaatggagccaggcatgaatttggtaaactcccccggacaggatgaaacagacacttctatcccttatatgatagatgcagacggagaaatccgttggattcttgattgggagaagtcggatgaacatcgatatatcggaatcgggtgcggattgatccggatgcagaacggacattatatgaccggtgacggaaaccatcatcggatggtggaggtggatatgatgggaaatacaattcataattgggatatgctggaaaggggatacactatgcatcatgccatatctcaagataagcaaggcaatatattagcgactgtgagcaagacttccgctaaaatcgccaatgggaaagatgtacggataaatgattttattattatgatggatcccgaaaaaggggagatattgcaagaatgggatttggtgcacatgttggattcggctcgctatggaatgaccgattatgatctaacctcagacccttttgcacaaagtgccagtaattgggcgcataataatggtatagtagagtggggagatgattatctggctacagctcgttatcaaggaatctttaagtttaataaagctggaggtattgagtggattatatcgcctcatgggtattggagagataaatacttgaaattattattgaatcctcttcatgccgacgggactcctattactgatcctgaggtgatagccggaacgaagaattgtgatgattttgaatggccttggggatgtcatacagctgtcccattacctaatggacacattctttatttcaataatggttatgggcgtaacttcaaactggattttacagacaggaaaaatatttatacttgcggcatagaatatgaagtggatgaagtgaaccgtacggttcgccaagtttggcagtatggaaaagagcgtggtgccgcgtattttactccggcccgttccggggtgcaatatttggaacaaacgggtaacagattaatttgtccgggaatgagcaatgtgcttagcaatggcaatcgtggtgcacgcgtgacagaagtagatcctgaaacacaggatgttgtgtttgagctggaattggaagatgcgatttaccagcgggtttaccgtatgtctctttatcctgagaaccaataa

SEQ ID NO: 2 - protein, 599 aa, unidentified
  source 1..599: note = Organism - Bacteroides vulgatus ATCC 8482
MKRKGYYLCSLLVMIFVFLSCRDEEQDCTQAMQDIQNQLKENDLLKSVSQEREQCVLLFESNKIIFPQELIQSVDVDAENWKTTLTFANGSTYVIPTLGTSIDNLILSSTVNPSGCNPLSASVVVKLPVLGRIKLIVHSKPGKHTPDVEYTFKDVGLKQNIPVLGLYPNYNNQITLIYTDLQGNERARSNLKLQTKTLESRRLPKEIRVVKAQYDRMEPGMNLVNSPGQDETDTSIPYMIDADGEIRWILDWEKSDEHRYIGIGCGLIRMQNGHYMTGDGNHHRMVEVDMMGNTIHNWDMLERGYTMHHAISQDKQGNILATVSKTSAKIANGKDVRINDFIIMMDPEKGEILQEWDLVHMLDSARYGMTDYDLTSDPFAQSASNWAHNNGIVEWGDDYLATARYQGIFKFNKAGGIEWIISPHGYWRDKYLKLLLNPLHADGTPITDPEVIAGTKNCDDFEWPWGCHTAVPLPNGHILYFNNGYGRNFKLDFTDRKNIYTCGIEYEVDEVNRTVRQVWQYGKERGAAYFTPARSGVQYLEQTGNRLICPGMSNVLSNGNRGARVTEVDPETQDVVFELELEDAIYQRVYRMSLYPENQ

SEQ ID NO: 3 - DNA, 1812 bp, synthetic construct
  source 1..1812: mol_type = other DNA
atggccatgaaacgcaaaggctattatctgtgcagcctgctggtgatgatttttgtgtttctgagctgccgcgatgaagaacaagattgcacccaagcgatgcaagatattcagaatcagctgaaagaaaacgatctgctgaaaagcgtgagccaagaacgcgaacagtgcgtgctgctgtttgaaagcaacaaaattatttttccgcaagaactgattcagagcgtggatgtggatgcggaaaactggaaaaccaccctgacctttgctaacggcagcacctatgtgattccgaccctgggcacgagcattgataacctgattctgagcagcaccgtgaacccgagcggctgcaacccgctgagcgcgagcgtggtggtgaaactgcccgtgctgggccgcattaaactgattgtgcatagcaaaccgggcaaacataccccggatgtggaatatacctttaaagatgtgggcctgaaacagaacattccggtgctgggcctgtatccgaactataacaatcagattaccctgatttataccgatctgcaaggcaacgaacgcgcgcgcagcaacctgaaactgcagaccaaaaccctggaaagccgccgcctgccgaaagaaattcgcgtggtgaaagcgcagtatgatcgcatggaaccgggcatgaacctggtgaacagcccgggccaagatgaaaccgatacgagcattccgtatatgattgatgcggatggcgaaattcgctggattctggattgggaaaaaagcgatgaacatcgctatattggcattggctgcggcctgattcgcatgcagaacggccattatatgaccggcgatggcaaccatcatcgcatggtggaagtggatatgatgggcaacaccattcataactgggatatgctggaacgcggctataccatgcatcatgcgattagccaagataaacaaggcaacattctggcgaccgtgagcaaaacgagcgcgaaaattgcgaacggcaaagatgtgcgcattaacgattttattattatgatggatccggagaaaggagaaattctgcaagaatgggatctggtgcatatgctggatagcgcgcgctatggcatgaccgattatgatctgacgagcgatccgtttgcgcagagcgcgagcaactgggcgcataacaacggcattgtggaatggggcgatgattatctggcgaccgcgcgctatcaaggcatttttaaatttaacaaagcgggaggcattgaatggattattagcccgcatggctattggcgcgataaatatctgaaactgctgctgaacccgctgcatgcggatggcaccccgattaccgatccggaagtgattgcgggcaccaaaaactgcgatgattttgaatggccgtggggctgccataccgcggtgccgctgccgaacggccatattctgtattttaacaacggctatggccgcaactttaaactggattttaccgatcgcaaaaacatttatacctgcggaatagaatatgaagtggatgaagtgaaccgcaccgtgcgccaagtgtggcagtatggcaaagaacgcggcgcggcgtattttaccccggcgcgcagcggcgtgcagtatctggaacagaccggcaaccgcctgatttgcccgggcatgagcaacgtgctgagcaacggcaaccgcggcgcgcgcgtgaccgaagttgatccggaaacccaagatgtggtgtttgaactggaactggaagatgcgatttatcagcgcgtgtatcgcatgagcctgtatccggaaaatcagtaagagctc

SEQ ID NO: 4 - protein, 601 aa, synthetic construct
MAMKRKGYYLCSLLVMIFVFLSCRDEEQDCTQAMQDIQNQLKENDLLKSVSQEREQCVLLFESNKIIFPQELIQSVDVDAENWKTTLTFANGSTYVIPTLGTSIDNLILSSTVNPSGCNPLSASVVVKLPVLGRIKLIVHSKPGKHTPDVEYTFKDVGLKQNIPVLGLYPNYNNQITLIYTDLQGNERARSNLKLQTKTLESRRLPKEIRVVKAQYDRMEPGMNLVNSPGQDETDTSIPYMIDADGEIRWILDWEKSDEHRYIGIGCGLIRMQNGHYMTGDGNHHRMVEVDMMGNTIHNWDMLERGYTMHHAISQDKQGNILATVSKTSAKIANGKDVRINDFIIMMDPEKGEILQEWDLVHMLDSARYGMTDYDLTSDPFAQSASNWAHNNGIVEWGDDYLATARYQGIFKFNKAGGIEWIISPHGYWRDKYLKLLLNPLHADGTPITDPEVIAGTKNCDDFEWPWGCHTAVPLPNGHILYFNNGYGRNFKLDFTDRKNIYTCGIEYEVDEVNRTVRQVWQYGKERGAAYFTPARSGVQYLEQTGNRLICPGMSNVLSNGNRGARVTEVDPETQDVVFELELEDAIYQRVYRMSLYPENQ

SEQ ID NO: 5 - protein, 617 aa, synthetic construct
MKYLLPTAAAGLLLLLAAQPAMAMAMKRKGYYLCSLLVMIFVLSCRDEEQDCTQAMQDIQNQLKENDLLKSVSQEREQCVLLFESNKIFPQELIQSVDVDAENWKTTLTFANGSTYVIPTLGTSIDNILISSTVNPSGCNPLSASVVKLPVLGIRKLIVHSKPGKHTPDVEYTFKDVGLKQNIPVIGLYPNYNNQITLIYTDLQGNERARSNLKLQTKTLESRRLDPKEIRVVKAQYDRMEPGMNLVNSPGQDETDTSIPYMIDADGEIRWLDWKSDEHRYIGCGCLIRMQNGHYMTGDGDNHRMVEVDMMGNTIHNWDMLERGTMHHAISQDKQGNILATVSKTSAKIANGKDVRINDDFIIMMDPEKGEILQEWDLVHMLDSARYGMTDYDLTSDPFAQSASNWAHNNGIVEWGDDYLATARYQGIFKNKAGGIEWIISPHGYWRDKYLKLLLNPLHADGTPITDPEVIAGTKNCDDDFEWPWGCHTAVPLPNGHILYFNNGYGRNFKLDFTDRKNIYTCGIEYEVDEVNRTVRQVWQYGKERGAAYFTPARSQVYLETGNRLICPGMSNVLSNGNRGARVTEVDPETQDVVFELELEDAIYQRVYRMSLYPENQ

SEQ ID NO: 6 - protein, 598 aa, synthetic construct
MKYLLPTAAAGLLLLLAAQPAMAMARDEEQDCTQAMQDIQNQLKENDLLKSVSQEREQCVLLFESNKIIFPQELIQSVDVDAENWKTTLTFANGSTYVIPTLGTSIDNLILSSTVNPSGCNPLSASVVKLPVLGIRKILIVHSKPGKHTPDVEYTFKDVGLKQNIPVLGLYPNYNNQITLIYTDLQGNERARSNLKLQTKTLESRRLDPKEIRVKAQYDRMEPGMNLVNSPGQDETDTSIPYMIDADGEIRWLDWKSDEHRYIGCGCLIRMQNGHYMTGDGDNHRMVEVDMMGNTIHNWDMLERGTMHHAISQDKQGNILATVSKTSAKIANGKDVRINDDFIIMMDPEKGEILQEWDLVHMLDSARYGMTDYDLTSDPFAQSASNWAHNNGIVEWGDDYLATARYQGIFKNKAGGIEWIISPHGYWRDKYLKLLLNPLHADGTPITDPEVIAGTKNCDDDFEWPWGCHTAVPLPNGHILYFNNGYGRNFKLDFTDRKNIYTCGIEYEVDEVNRTVRQVWQYGKERGAAYFTPARSGVYLETGNRLICPGMSNVLSNGNRGARVTEVDPETQDVVFELELEDAIYQRVYRMSLYPENQ

SEQ ID NO: 7 - protein, 576 aa, synthetic construct
MARDEEQDCTQAMQDIQNQLKENDLLKSVSQEREQCVLLFESNKIFPQELIQSVDVDAENWKTTLTFANGSTYVIPTLGTSIDNILISSTVNPSGCNPLSASVVKLPVLGIRKILIVHSKPGKHTPDVEYTFKDVGLKQNIPVIGLYPNYNNQITLIYTDLQGNERARSNLKLQTKTLESRRLPKEIRVVKAQYDRMEPGMNLVNSPGQDETDTSIPYMIDADGEIRWLDWKSDEHRYIGICGCLIRMQNGHYMTGDGDNHRMVEVDMMGNTIHNWDMLERGTMHHAISQDKQGNILATVSKTSAKIANGKDVRINDDFIIMMDPEKGEILQEWDLVHMLDSARYGMTDYDLTSDPFAQSASNWAHNNGIVEWGDDYLATARYQGIFKNKAGGIEWIISPHGYWRDKYLKLLLNPLHADGTPITDPEVIAGTKNCDDDFEWPWGCHTAVPLPNGHILYFNNGYGRNFKLDFTDRKNIYTCGIEYEVDEVNRTVRQVWQYGKERGAAYFTPARSQVYLEQTGNRLICPGMSNVLSNGNRGARVTEVDPETQDVVFELELEDAIYQRVYRMSLYPENQ

SEQ ID NO: 8 - protein, 60 aa, synthetic construct
TDPEVIAGTKNCDDFEWPWGCHTAVPLPNGHILYFNNGYGRNFKLDFTDRKNIYTCGIEY

SEQ ID NO: 9 - protein, 57 aa, synthetic construct
TCNENGLCENSDDFETYTQHTAWLSSKGTLITFDNGDGRHLEQPALPTMKYSRFVEY

SEQ ID NO: 10 - protein, 60 aa, synthetic construct; identical to SEQ ID NO: 8

SEQ ID NO: 11 - protein, 60 aa, synthetic construct; identical to SEQ ID NO: 8

SEQ ID NO: 12 - protein, 60 aa, synthetic construct; identical to SEQ ID NO: 8

SEQ ID NO: 13 - DNA, 27 bp, synthetic construct
  source 1..27: mol_type = other DNA
ccatggcccgcgatgaagaacaagatt

SEQ ID NO: 14 - DNA, 24 bp, synthetic construct
  source 1..24: mol_type = other DNA
gagctcttactgattttccggata